(19) United States
(12) Patent Application Publication
(10) Pub. No.: US 2020/0038509 A1
(43) Pub. Date: Feb. 6, 2020

(54) RADIOFREQUENCY FIELD HYPERTHERMIA AND SOLID TUMOR IMMUNOMODULATION

(71) Applicant: Baylor College of Medicine, Houston, TX (US)

(72) Inventors: Stuart James Corr, Houston, TX (US); Jared M. Newton, Houston, TX (US); Steven A. Curley, Missouri City, TX (US); Andrew G. Sikora, Houston, TX (US)

(21) Appl. No.: 16/341,414

(22) PCT Filed: Oct. 13, 2017
(86) PCT No.: PCT/US2017/056621

§ 371 Date: Apr. 11, 2019

Related U.S. Application Data
(60) Provisional application No. 62/408,433, filed on Oct. 14, 2016.
(60) Provisional application No. 62/408,583, filed on Oct. 14, 2016.

(51) Int. Cl. A61K 41/00; A61N 1/40; A61K 38/20; A61K 38/21; A61K 38/19; A61P 35/00

(52) U.S. Cl. CPC A61K 41/0052; A61N 1/403; A61K 38/2013; A61K 38/208; A61P 35/00; A61K 38/20; A61K 38/212; A61K 38/193; A61K 38/191; A61K 38/2073

(57) ABSTRACT

Embodiments of the disclosure concern methods and compositions for treating cancer in an individual that includes combination therapy comprising radiofrequency therapy and immunotherapy. In specific embodiments, utilization of the radiofrequency therapy invokes the immune system of the individual to provide an environment at the tumor that is receptive to native immune system components of the individual, and/or exogenously provided immune system components. In specific cases, the radiofrequency therapy enhances T cell localization to the tumor.

This application claims priority to U.S. Provisional Patent Application Ser. No. 62/408,433, filed Oct. 14, 2016, and also to U.S. Provisional Patent Application Ser. No. 62/408,583, filed Oct. 14, 2016, both of which applications are incorporated by reference herein in their entirety.

### STATEMENT REGARDING FEDERALLY SPONSORED RESEARCH OR DEVELOPMENT

This invention was made with government support under T32GM088129 awarded by National Institutes of Health. The government has certain rights in the invention.

### TECHNICAL FIELD

Embodiments of the disclosure include at least the fields of cell biology, immunology, molecular biology, and medicine.

### BACKGROUND

Many tumors are resistant to immune attack because they are relatively impermeable to infiltration of effector immune cells with antitumor activity. Additionally, immune-suppressive cells within the tumor limit effectiveness of effector immune cells. Thus, therapies which alter the balance of immune-suppressive and effector immune cells within the tumor have the potential to induce effective anti-tumor immunity, and enhance the efficacy of immunotherapeutic approaches.

The present disclosure provides a solution for a long-felt need in the art to utilize radiofrequency therapy effectively in an immune environment for therapy.

### BRIEF SUMMARY

Embodiments of the disclosure encompass methods and compositions that utilize radiofrequency (RF) therapy to favorably enhance the immune environment at a tumor site. In particular aspects of RF therapy, the ability of RF to modulate the cellular immune microenvironments at a tumor site provides an opportunity to combine the RF therapy with one or more compounds that can be therapeutically effective at the tumor site that otherwise would have been less effective therapeutically.

In particular embodiments, the combination of RF therapy and other immunotherapy strategies would be provided to the individual. This would allow the RF therapy to induce an intra-tumoral inflammatory response such that the immunotherapy in combination with the RF therapy will exhibit a combinatorial effect. This disclosure encompasses methods for priming a tumor immunologically using RF exposure that serves to enhance a number of other immunotherapeutic strategies, such as immune checkpoint inhibitor, radiation, chemotherapeutic regimens, INOS inhibitor efficacy, chimeric antigen receptor (CAR) T-cell therapies, other adoptive immune cell therapies, cancer vaccine strategies, immune costimulatory receptor agonist antibodies, and/or oncolytic virotherapies, for example. Methods of the disclosure show that RF therapy can be used to overcome the highly non-active and immunosuppressive state of a tumor's immune microenvironment, thereby allowing other therapies, such as immunotherapy, to be effective. Specific methods of this disclosure show that RF can favorably enhance the immunologic state of solid tumor. In particular, this disclosure shows that RF treatment can induce a transient T cell-specific infiltration into the tumor, transient migration of macrophages out of the tumor (which can then prime a more significant tumor-specific T-cell response), higher activation states of tumor residing T-cells, and an immunologically active state in distal tumors. Embodiments of the disclosure include methods for deliberately increasing tumor T cell infiltration and activation by exposing the tumor site to RF therapy and then exposing the tumor site to one or more immunotherapies to exploit the immunomodulated environment at the tumor produced by the RF therapy.

Embodiments of the disclosure encompass methods and compositions that utilize radiofrequency (RF) therapy to reverse immunosuppressive mechanisms at a tumor site. Embodiments of the disclosure utilize RF field hyperthermia to manipulate immunomodulation at a tumor. In particular aspects, the ability of RF to modulate immunogenicity at a tumor site provides the opportunity to combine the RF therapy with one or more compounds that can be therapeutically effective at the tumor site that otherwise without the RF therapy would not have been therapeutically effective.

In particular embodiments, the combination of RF therapy and immunotherapy is provided to the individual for the purpose of the RF therapy inducing an intra-tumoral inflammatory response such that the immunotherapy in combination with the RF therapy will exhibit a combinatorial effect. The disclosure encompasses methods of intentionally priming a tumor with immunomodulation (such as immunomodulation effected by RF therapy) that enhances an immunotherapy, such as enhances therapeutic immune checkpoint inhibitor and/or INOS inhibitor efficacy (as examples only). Methods of the disclosure overcome an immunosuppressive microenvironment at a tumor by reversing the immunosuppression at the tumor by using RF therapy on the tumor, thereby allowing another therapy such as immunotherapy to be effective. Tumor immunogenicity becomes less robust upon use of methods of the disclosure that utilize RF therapy to modulate an immune response at the tumor, such as by inducing a transient T cell-driven response to infiltrate the tumor with at least helper T-cells, CD8+ cytotoxic T lymphocytes, natural killer (NK cells), M1-polarized (anti-tumor) macrophages, and antigen-presenting dendritic cells, or a combination thereof. Embodiments of the disclosure include methods of deliberately increasing T cell infiltration at a tumor site by exposing the tumor site to RF therapy to enhance T cell infiltration and exposing the tumor site to one or more immunotherapies to exploit the immunomodulated environment at the tumor produced by the RF therapy.

Agents utilized in standard-of-care cancer therapy, including radiotherapy and chemotherapy (e.g. the cytotoxic agent cyclophosphamide), have well-known immune effects and may be enhanced in combination with RF therapy.

In one embodiment, there is a method of treating an individual for cancer, comprising the step of delivering to the individual a therapeutically effective amount of radiofrequency therapy (RF) and immunotherapy. In a specific embodiment, the immunotherapy comprises one or more immune checkpoint inhibitors, therapeutic vaccine targeting tumor antigens; innate immune-stimulating molecules or biologics; adaptive immune-stimulating molecules or biologics; oncolytic virotherapy agents, monoclonal antibodies or other agents targeting positive immune costimulatory molecules; adoptive cellular therapy; chimeric antigen receptor (CAR) T-cells; cancer vaccine; or a combination thereof. The immune checkpoint inhibitor may be ones targeting CTLA-4, PD1, PD-L1, PD-L2, TIM-3, B7-H3, IDO, or a combination thereof. The immunotherapy may comprise an INOS inhibitor, such as LNIL, L-NMMA 1400 W dihydrochloride, AR-C 102222, AMT hydrochloride, S-Isopropylisothiourea hydrobromide, Aminoguanidine hydrochloride, BYK 191023 dihydrochloride, EIT hydrobromide, (S)-Methylisothiourea sulfate or a combination thereof. In some cases, the immunotherapy comprises one or more immune-stimulating agonist antibodies or other molecules, targeting GITR, OX-40, IL-2, IL-12, IL-18, IFNα, IL-11, GM-CSF, G-CSF, or other positive costimulatory molecules that may be targeted by agonist monoclonal antibodies or other means.

In certain cases, the RF therapy and the immunotherapy are delivered to the individual at the same or different times. In specific cases the RF therapy is delivered to the individual before the immunotherapy, or it may be delivered to the individual after the immunotherapy. In specific embodiments, the RF therapy is delivered to the individual multiple times and/or the immunotherapy may be delivered to the individual multiple times. In some cases, the individual is given a therapy other than the RF and the immunotherapy for the cancer, such as another immunotherapy, surgery, chemotherapy, radiation, hormone therapy, or a combination thereof. In specific cases, the individual is given surgery prior to or after the delivery of the RF and immunotherapy. The RF therapy may be given to the individual for at least 5, 10, 15, 20, 30, 35, 40, 45, 50, 55, or 60 minutes in duration. In some cases, the temperature that is generated at a desired location to which the radiofrequency is directed is between 37° C. and 45° C.

The present disclosure details the use of non-invasive radiofrequency field therapy as an effective modality for altering the immune microenvironment in solid tumor cancers. The details encompassed in this disclosure are unique to the pre-existing uses for radiofrequency therapy that have primarily focused on enhancing perfusion or using hyperthermia as the sole source of treatment of solid tumors.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiments disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

FIG. 1 shows mouse treatment including the RF set-up, copper tape extremity grounding, and temperature monitoring using infra-red camera;

FIG. 2 shows one example of an experimental design for a immune microenvironment experiment using RF and IL-12 in Balb/c mice with 4T1 (breast cancer) tumors;

FIG. 3 provides the results from the study in FIG. 2, showing that RF promotes a significant increase in CD4+ T-cell infiltration into the tumor and an increase in the cytotoxic variety of CD4+ T-cells (cells that contain Granzyme B);

FIG. 4 provides more results from the study in FIG. 2, showing an upregulation of iNOS expression and PD-L1 expression (both immunosuppressive markers indicative of an immunologically active tumor microenvironment) in the non-immune tumor cells (CD45 negative cells) following RF.

FIG. 5 demonstrates that in the spleen of the mice in the microenvironment experiment in FIGS. 2-4, there was no induced changes via RF, indicating that RF is acting locally to change the immune microenvironment in the tumor without inducing significant systemic immune changes.

FIG. 6 demonstrates an experimental set-up for a tumor growth study wherein Balb/c mice with 4T1 (breast cancer) tumors were either RF-treated (41° C., 30 mins) or control treated through “No-Heat Control (NHC)” conditions. Image shows a nude mouse undergoing NHC treatment.

FIG. 7 shows results from the tumor growth study (n=10 per group) detailed in FIG. 6, showing that RF-treated mice had a significant growth rate increase, demonstrating that RF is inducing intratumoral inflammation within these tumors, thereby causing them to appear larger due to enhanced infiltration of immune cell mediators.

FIG. 8 provides a similar tumor growth experiment as in FIG. 7 performed in Athymic Nude Balb/c mice (which lack functional T-cells) with 4T1 tumors, which shows no difference in tumor size for RF-treated and untreated mice, indicating that T-cells are a primary mediator of the RF induced intra-tumoral inflammation

FIG. 9 illustrates a schematic showing an experimental design for a second immune microenvironment experiment in Balb/c mice with 4T1 (breast cancer) tumors in which singlet tumor mice either did or did not receive RF, and doublet tumor mice only received RF treatment on one tumor. Tissue was then processed at various time point post-treatment (24, 48, and 120 hours).

FIG. 10 shows the tumor growth curves from the singlet tumor microenvironment treated mice between when they were RF-treated (day 12), and when they were sacked for tissue processing, demonstrating the transient induction of tumor inflammation by RF treatment

FIG. 11 provides growth curves for the dual tumor mice following a single RF-treatment (day 12) up until the day of tissue processing for the microenvironment study, demonstrating the induction of inflammation in both tumors equally which results in no size differential

FIG. 12 illustrates the flow cytometry gating strategy for the lymphocyte staining panel that was used for the immune microenvironment studies.

FIG. 13 illustrates the flow cytometry gating strategy for the myeloid staining panel that was used for the immune microenvironment studies.

FIG. 14 provides results from a second microenvironment study detailed in FIG. 9, showing the changes in CD4+ T-cells in the tumor at 24, 48, and 120 hours. This demonstrates an increase in tumor infiltration of CD4+ T-cells 24 hours post-RF treatment and an increase in the cytotoxic variety of CD4+ T-cells (CD4+ T-cells that contain perforin, granzyme B, and IFNg) at 24 hours post-RF treatment.

FIG. 15 provides more results from a second microenvironment study detailed in FIG. 9, it demonstrates that although there was no significant changes in tumor infiltrating CD8+ T-cell at any of the monitored time points post-RF treatment, there was an increase in “activated” CD8+ T-cells at 24 hours post-RF treatment (CD8+ T-cells that contain perforin and IFNg).

FIG. 16 provides more results from a second microenvironment study detailed in FIG. 9, it shows that by 120 hours post-RF, there is a significant decrease in tumor cell viability in the RF-treated group.

FIG. 17 provides more results from a second microenvironment study detailed in FIG. 9, show that RF treatment induces a significant decrease in intra-tumoral macrophages at 24 hours post-RF, which return to normal levels by 48 hours. The bottom graphs shows that there is a significant increase in myeloid derived suppressor cells in the RF-treated tumors at 120 hours post-RF treatment.

FIG. 18 provides more results from a second microenvironment study detailed in FIG. 9, specifically showing the immune changes in the dual tumor mice. Each plot shows the cellular percentages for the singlet tumor RF-treated mice, singlet tumor NHC mice, dual tumor mice RF-treated side, dual tumor mouse non-RF treated side (left to right). All show the tumoral changes at 24 hours post-RF treatment except the bottom right graph which shows viability changes at 120 hours post-RF treatment. These results show a highly suppressive tumor microenvironment in the non-RF treated tumor in the dual mice (high levels of CTLA-4 and PD-L1), indicating that RF treatment of a primary tumor can induce immune responses in a distal non-RF treated tumor.

FIG. 19 provides more results from a second microenvironment study detailed in FIG. 9, and shows the immune changes in the tumor draining lymph nodes at 120 hours post-RF for both the singlet and dual tumor mice.

FIGS. 20A, 20B, and 20C concern RFT set-up and temperature monitoring. 20A) Schematic depicting capacitively-coupled RF transmitting and receiving head showing mouse orientation and copper blanket shielding. 20B) Image of mouse grounding and shielding showing exposed tumor (green arrow) and rectally inserted fiber-optic probe (red arrow) used for systemic temperature monitoring. Representative graph to the right shows systemic temperature measurement for a single mouse during an entire RFT session. 20C) Image from infrared camera showing exposed tumor used for tumor surface temperature monitoring. Representative graph to the right shows tumor surface temperature measurement for a single mouse during an entire RFT session. (FIG. 24A shows cumulative treatment systemic and tumor surface heating curves).

FIGS. 21A-21D demonstrate that consecutive-dose RFT induces T-cell dependent tumor growth effect. 4T1 tumor volume following multiple consecutive RFT (41° C., 30 mins; date of treatment indicated by black arrows) in either 21A) wild-type Balb/c mice or 21B) athymic nude Balb/c mice (n=10/group). 21C) Representative H&E histology images of control (top) and RF-treated (bottom) tumors at termination (black arrow denotes necrotic fraction), with quantified necrotic fraction graph showing percent tumor necrosis between RF-treated and control mice (n=5-6/group). 21D) Representative IHC images showing Ki67 expression comparison between control (top) and RF-treated (bottom) tumors. Error bars represent SEM. (See FIGS. 26A and 26B for complete image set). (*p<0.05, **p<0.01, ***p<0.001).

FIGS. 22A-22D show immune microenvironment time-course analysis following single-dose RFT. 22A) 4T1 tumor volume in Balb/c mice following single RFT (41° C., 30 mins; day of treatment indicated by black arrow) which were terminated for microenvironment analysis at either 24 hrs (left), 48 hrs (middle), or 120 hrs (right) post-RFT (n=5/group). 22B) Representative flow cytometry scatter plots showing the increase in CD4+ and CD8+ T cell populations within tumor between a control and RF-treated mice 24 hours post-RFT (top) and within tumor-draining lymph node between 24 hrs post-RFT and 120 hrs post-RFT mice (bottom). 22C) Cumulative plots of CD4+ T cell percentages (top row) and CD8+ T cell percentages (bottom row) showing changes in T cell percentages among total viable cells 24 hrs post-treatment for tumor (left column) and changes 24, 48, and 120 hrs post-treatment in tumor-draining lymph node (right column). (n=3-10/group). 22D) Control vs. RF-treated tumor percentage of MDSC (CD11b+/Gr1+) 120 hrs post-treatment with representative flow plots (left) and cumulative plots showing percentages among total viable cells (right) (n=5-10/group). Each point represents an individual mouse percentage and all percentages are out of total viable cells. Error bars represent SEM. (*p<0.05, **p<0.01, ***p<0.001).

FIGS. 23A-23B provide blood plasma cytokine panel time-course analysis following single-dose RFT. At 24, 48, or 120 hrs after a single RFT blood plasma was analyzed for 25 cytokines. 23A) Cytokine plasma concentration fold-change of RF-treated mice at 24, 48, or 120 hours post-RFT compared to control mice. Fold changes for each analyte were calculated by taking the ratio of average plasma concentrations of RFT-mice and control mice for each independent time-point (i.e. [G-CSF]_{24 hr RF}/[G-CSF]_{24 hr control}) (n=5/group). 23B) Plasma concentration in pg/mL of IL-6 (top) and MIP2 (bottom) comparing control and RF-treated mice 24 hrs post-RFT (n=5/group). Error bars represent SEM. (*p<0.05, **p<0.01, ***p<0.001).

FIGS. 24A-24C show that RFT promotes consistent and safe intratumoral hyperthermia. 24A) Representative systemic temperature measurements from rectally inserted fiber-optic probe (left) and tumor surface temperature measurement from IR camera for RFT mice bearing 4T1 tumors showing changes during an entire treatment duration (n=10 mice, 2 different treatment days). 24B) Mouse weight changes in grams following multiple treatments between RFT and control-treated mice (n=10/group; for schedule see FIG. 21A). 24C) Mouse lung weights (mg) after multiple treatments between RFT and control-treated mice (n=10/group; each dot represents an individual mouse lung weight).

FIGS. 25A and 25B show that RFT induced changes in T-cell activation and macrophages. 25A) Cumulative dot plots showing perforin (left) and IFNγ (right) median fluorescent intensity among tumor dwelling CD8+ T-cell at 24, 48, and 120 hours post-treatment (n=5/group). 25B) Tumor macrophage percentages among total viable cells at 24, 48, and 120 hours post-treatment (left; n=5/group). Plasma concentration in pg/mL of G-CSF comparing control and RF-treated mice 24 hrs post-RFT (right; n=5/group)

FIGS. 26A and 26B provide H&E and Ki67 histology of control and RFT tumors. Full image set showing 26A) H&E staining and 26B) Ki67 immunohistochemical staining following multiple treatments, either RFT or control (see FIG. 21A for example of treatment schedule).

### DETAILED DESCRIPTION

As used herein the specification, “a” or “an” may mean one or more. As used herein in the claim(s), when used in conjunction with the word “comprising”, the words “a” or “an” may mean one or more than one. As used herein “another” may mean at least a second or more. Still further, the terms “having”, “including”, “containing” and “comprising” are interchangeable and one of skill in the art is cognizant that these terms are open ended terms. Some embodiments of the disclosure may consist of or consist essentially of one or more elements, method steps, and/or methods of the disclosure. It is contemplated that any method or composition described herein can be implemented with respect to any other method or composition described herein.

Radiofrequency (RF) field treatment has previously been reported to induce alterations in cancer cell phenotype (Ware et al. 2015); re-sensitization of chemo-resistant cancer cells; enhancement of nanoparticle extravasation in solid tumors (Lapin et al. 2016); an increase in vascular perfusion in solid tumors; and an increase in drug uptake in solid tumors. The present disclosure concerns RF therapy for inducement of an anti-tumor immune response.

In particular, the present disclosure concerns methods and compositions for using RF therapy for immunomodulation of a solid tumor. In specific embodiments, methods and compositions concern RF immunomodulation, wherein RF treatment can enhance one or more immune system mechanisms at a localized site, such as a tumor site, including adaptive immune system mechanisms and/or innate immune system mechanisms. Embodiments of the disclosure include using RF for immunologic tumor priming that could then enhance the efficacy of one or more immunotherapy strategies, for example RF immunodulation that allows enhancement of immune checkpoint inhibitor treatment efficacy at a tumor site.

In embodiments of the disclosure, RF induces a transient intra-tumoral inflammatory response that is dependent on T-cells, in specific cases through increased infiltration of at least helper T-cells. One can utilize serum cytokine analysis, histology and/or chemokine arrays to verify the type of inflammatory response. In addition, one could dissociate the tumor to a single-cell suspension, as is familiar to any practitioner skilled in the art, and use flow cytometry for immunologic markers to profile the immunocyte population(s) present in the tumor. Also gene expression analysis, using commonly employed methods, including but not limited to PCR array; RNA-sequencing; and nanostring analysis, can be used to identify signatures of immune effector and immunosuppressive response. Cell populations may also be analyzed by flow cytometry, and activity of specific subpopulations of immune system cells can be measured in cytotoxicity, cell mobility, or other functional assays.

In specific embodiments, RF is utilized in combination with immunotherapy, such as one or more immune checkpoint inhibitors and/or other immunomodulators to exhibit a combinatorial effect. In specific embodiments, the combination of RF and one or more immune checkpoint inhibitors or other immunomodulators exhibits an additive or synergistic effect upon combinatorial use at a tumor site.

In particular embodiments, RF increases hyperinflammation localized to a tumor. In particular aspects, RF induces T cells to a tumor site. In certain embodiments, RF increases infiltration of T cells to a tumor, such as CD4 T cells. In specific embodiments, RF clears a tumor of macrophages. Embodiments of the disclosure encompass RF to alter the cell population at a tumor site, including changing the cell population of immune cells at a tumor site. Following use of methods of the disclosure, the number of cytotoxic immune cells are increased at a tumor site.

RF therapy may be utilized with one or more immunotherapies of any kind so that the RF therapy generates a localized environment at the site that is receptive to the immunotherapy, thereby allowing the immunotherapy to be effective when in the absence of the RF therapy the immunotherapy would have been less effective or not effective. Embodiments of the disclosure encompass a therapeutic effect from one or more immune checkpoint inhibitors, in at least certain cases. In specific embodiments, the immunotherapy may comprise one or more immune checkpoint inhibitors or other immunomodulators. The immune checkpoint inhibitor may be antibodies targeting CTLA-4, PD1, PD-L1, PD-L2, TIM-3, B7-H3, IDO, and/or other immune checkpoint molecules. Additionally, immune-stimulating molecules such as GITR, OX-40, IL-2, IL-12, IL-18, IFNα, IL-11, GM-CSF, G-CSF, and other positive costimulatory molecules may be targeted by agonist monoclonal antibodies or other means. In alternative cases, the immunotherapy is not an immune checkpoint inhibitor. In specific cases, the immunotherapy is an INOS inhibitor, such as LNIL, L-NMMA, 1400 W dihydrochloride, AR-C 102222, AMT hydrochloride, S-Isopropylisothiourea hydrobromide, Aminoguanidine hydrochloride, BYK 191023 dihydrochloride, EIT hydrobromide, (S)-Methylisothiourea sulfate or a combination thereof. The immunotherapy may be cyclosphosphamide, in certain cases. Additional immunotherapeutic approaches that may be used in combination with the method include at least one or more of the following: therapeutic cancer vaccines targeting various classes of tumor antigens; innate immune stimulation by targeting pattern recognition receptors, such as agonists of Toll-like receptors 9, 7, 8, 4, or other innate immune sensing molecules; adoptive cellular immunotherapy such as infusion of cultured tumor infiltrating lymphocytes (TIL) or chimeric antigen receptor (CAR) T cells or NK-T cells, or natural killer (NK) cells or dendritic cells. The immunotherapy may comprise combinations of cyclophosphamide, an iNOS inhibitor, cisplatin, other platinum drugs, nucleotide analogues, methotrexate, taxanes, and/or ionizing radiation.

The cancer being treated by the combination therapy of the disclosure includes any cancer. In specific embodiments the cancer comprises solid tumors. In specific cases, the cancer is not blood-borne. In specific embodiments, the cancer is a primary cancer, metastatic cancer, resistant cancer, recurrent cancer, and so forth. The cancer may be of the brain, lungs, breast, prostate, pancreas, skin, ovary, kidney, liver, stomach, colon, head and neck, gall bladder, testes, cervix, uterus, bladder, bone, thyroid, blood, endometrium, spleen, pituitary gland, and so forth.

Combination therapy of the present disclosure includes RF therapy and one or more immunotherapies. The dosing regimen of the combination therapy may include single doses of both components of the combination therapy, single doses of one of the components of the combination therapy but multiple doses of the other component of the combination therapy, or multiple doses of both components of the combination therapy. In specific embodiments, multiple doses of both components of the combination therapy are provided to an individual in need thereof over a specific duration of time, such as over a duration of a week, a month, several months, a year, or several years, for example. In some cases, the RF component of the combination therapy is given to the individual prior to the immunotherapy component of the combination therapy, in other cases the RF component of the combination therapy is given to the individual after the immunotherapy component, and in some cases they are given simultaneously.

In some cases, the combination therapy that includes both RF and immunotherapy is given to the individual in addition to another therapy, such as another immunotherapy, surgery, chemotherapy, radiation, hormone therapy, or a combination thereof. In specific embodiments, a tumor is resected in an individual that is also receiving, has received, or will receive the combination therapy. In specific cases, an individual has one or more tumors resected prior to and/or subsequent to delivery of one or more doses of the combination therapy. In specific aspects, the combination therapy is given to the individual 1, 2, 3, 4, 5, 6, 7, 8, 9, 10, or more times prior to and/or subsequent to a tumor resection.

In particular embodiments, an individual is given the combination therapy and the size of the tumor is monitored. In some cases for solid tumors, one may periodically biopsy the tumor and assess the quantity and character of its immune infiltrate by immunohistochemistry, flow cytometry, or other methods. Dynamic contrast-enhanced imaging, including arterial phase computed tomography or magnetic resonance imaging, can be used to assess changes in tumor blood supply and document areas of tumor necrosis or regions with reduced perfusion demonstrating cancer cell injury. Tumor viability can also be measured by changes in metabolic imaging, including but not limited to positron emission tomography. In specific embodiments, the size of a tumor in the individual is ascertained following delivery of the combination therapy and in addition or alternative to the size being determined prior to the therapy. Any procedure to analyze the size of the tumor may be utilized, including, for example, FDG positron emission tomography (FDG-PET), magnetic resonance imaging (MRI), optical imaging, bioluminescence imaging (BLI), fluorescence imaging (FLI), and so forth.

Identification of the area to be treated in the individual may encompass any suitable means for determining location of one or more tumors. In specific embodiments, the area to be treated may be identified by palpitation, x-ray, endoscopy, magnetic resonance imaging, CT scan, radionuclide scan, positron emission tomography scan, and/or ultrasound, for example. In particular embodiments, one can image the area of interest such that the radiofrequency therapy may be focused accurately on the tumor.

### I. Radiofrequency Therapy Embodiments

Embodiments of the disclosure include providing to an individual in need thereof radiofrequency under sufficient conditions to induce an anti-tumor immune response at a localized site, such as by using the application of a non-invasive radiofrequency field, including one generated by a radiofrequency signal between a transmission head and a reception head that is different from the transmission head. One can configure the transmission and reception heads on opposite sides of a desired target of the individual for treatment (such as a tumor site(s) or the whole body) and irradiate the site(s) between the transmission and reception heads with a radiofrequency field to kill, damage, or induce immune responses against the target cells from the interaction of the radiofrequency field with the cancer cells.

In specific embodiments, a non-invasive radiofrequency therapy system comprises a radiofrequency transmitter in communication with a transmission head and a radiofrequency receiver in communication with a reception head. The communication may be direct electrical, optical, and electromagnetic connections and indirect electrical, optical, and electromagnetic connections. That is, two devices are in communication if a signal from one is received by the other, regardless of whether the signal is modified by some other device. In an example of a non-invasive radiofrequency therapy system, the radiofrequency transmitter generates a radiofrequency signal at a frequency for transmission via the transmission head. In some cases, the radiofrequency transmitter has controls for adjusting the frequency and/or power and/or amplitude modulation of the generated radiofrequency signal and/or may have a mode in which a radiofrequency signal at a predetermined frequency and power are transmitted via the transmission head. In some cases, the radiofrequency transmitter provides a radiofrequency signal with variable amplitudes, pulsed amplitudes, multiple frequencies, etc.

In particular embodiments, the radiofrequency receiver is in communication with the reception head and is tuned such that at least a portion of the reception head is resonant at the frequency of a radiofrequency signal transmitted via the transmission head. As a result, the reception head receives a radiofrequency signal that is transmitted via the transmission head. In specific cases, the transmission head and reception head are arranged proximate to and on either side of a general target area, such as an area that has the tumor to be treated. The transmission head and reception head may be insulated from direct contact with the general target area, in certain aspects. In specific cases, the transmission head and reception head are insulated by means of an air gap, although in some cases it is an insulating layer or material, such as, for example, Teflon®. One can include an insulation area on the heads, allowing the heads to be put in direct contact with the general target area. The transmission head and the reception head may include one or more plates of electrically conductive material such as gold, silver, or copper.

The target tumor absorbs energy through its inherent dielectric and electrical properties and is warmed as the radiofrequency signal travels through the target tumor area that is desired to be treated by inducing hyperthermia. The more energy that is absorbed by an area, the higher the temperature increase in the area. In specific embodiments, the target area is heated to between 37° C.-45° C., for example. The target area may be heated to 37° C., 38° C., 39° C., 40° C., 41 ° C., 42° C., 43° C., 44° C., or 45° C. In some cases, the target area is heated in the range of 37° C.-45° C., 38 ° C.-44° C., 38 ° C.-43° C., 38 ° C.-42° C., 38 ° C.-41° C., 38 ° C.-40° C., 39 ° C.-45° C., 39 ° C.-44° C., 39 ° C.-43° C., 39 ° C.-43° C., 39 ° C.-42° C., 39 ° C.-41° C., 39 ° C.-40° C., 40 ° C.-45° C., 40 ° C.-44° C., 40° C.-43° C., 40° C.-42° C., 40° C.-41° C., 41° C.-45° C., 41° C.-44° C., 41° C.-43° C. 41° C.-42° C., 42° C.-45° C., 42° C.-44° C., 43° C.-45° C., 43° C.-44° C., 44 ° C. -45° C. and so forth. In particular embodiments, the temperature to substantially damage the targeted tumor cells is sufficient to kill the tumor cells without damaging or substantially damaging surrounding normal cells and without tissue burn, for example.

Energy absorption in the target tumor area can be increased by increasing the radiofrequency signal strength, which increases the amount of energy traveling through the area. One method of inducing a higher temperature in a specific target tumor area includes using a reception head that is smaller than the transmission head. The smaller reception head picks up more energy due to the use of a high-Q resonant circuit. In specific embodiments, the temperature is monitored, for example by MRI thermography.

In specific embodiments, the radiofrequency power is determined by the type of system being employed. For example, for a portable system one may utilize 0-200 watts (W). In particular cases wherein the system is not portable, one may employ, e.g., from 700 W-1500 W to maintain a localized electric-field of strength 0-90 kV/m.

In certain embodiments, one or more particular wavelengths are employed. In specific cases, a frequency of 13.56 MHz is employed. Other examples are 1 MHz, 6.78 MHz, 8 MHz, 27.12 MHz, 40.68 MHz, 128 MHz, etc. In specific embodiments, a frequency range of 100 kHz to 1 GHz is employed. Other examples of ranges include 250 kHz-1 GHz, 500 kHz-1 GHz, 1000 kHz-1 GHz, 10,000 kHz-1 GHz, 100,000 kHz-1 GHz, 1 MHz-1 GHz, 10 MHz-1 GHz, 100 MHz-1 GHz, 500 MHz-1 GHz, 10 MHz-50 MHz, 10 MHz-100 MHz, 10 MHz-250 MHz, 10 MHz-500 MHz, and so forth. In some embodiments, radiofrequency energy from the kilohertz to the low gigahertz range can cause effects in malignant tumor microenvironments, and these effects can be accentuated by using pulsed or amplitude modulated radiofrequencies.

The frequency and duration of exposure of the non-invasive radiofrequency therapy to the individual may be optimized for the individual, type of cancer, gender, size of the individual, and so forth. In specific embodiments, the individual may be provided with the non-invasive RF therapy and the one or more immunotherapies once or more than once during a particular period of treatment. In specific embodiments, the RF therapy and one or more immunotherapies are provided to the individual over the course of 1-7, 1-6, 1-5, 1-4, 1-3, 1-2, 2-7, 2-6, 2-5, 2-4, 2-3, 3-7, 3-6, 3-5, 3-4, 4-7, 4-6, 4-5, 5-7, 5-6, or 6-7 days or over the course of 1-4, 1-3, 1-2, 2-4, 2-3, or 3-4 weeks. However, in some cases the radiofrequency therapy and one or more immunotherapies are provided to the individual over the course of 1-12, 1-11, 1-10, 1-9, 1-8, 1-7, 1-6, 1-5, 1-4, 1-3, 1-2, 2-12, 2-11, 2-10, 2-9, 2-8, 2-7, 2-6, 2-5, 2-4, 2-3, 3-12, 3-11, 3-10, 3-9, 3-8, 3-7, 3-6, 3-5, 3-4, 4-12, 4-11, 4-10, 4-9, 4-8, 4-7, 4-6, 4-5, 5-12, 5-11, 5-10, 5-9, 5-8, 5-7, 5-6, 6-12, 6-11, 6-10, 6-9, 6-8, 6-7, 7-12, 7-11, 7-10, 7-9, 7-8, 8-12, 8-11, 8-10, 8-9, 9-12, 9-11, 9-10, 10-12, 10-11, or 11-12 months. An individual may be provided the non-invasive radiofrequency therapy upon recurrence of a cancer from remission or upon having another type of cancer altogether, in which case the combination radiofrequency/immunotherapy deliveries may be employed years apart. Thus, in some cases the radiofrequency therapy and one or more immunotherapies are provided to the individual over the course of 1-5, 1-4, 1-3, or 1-2 years.

The duration of exposure to radiofrequency may be of any suitable time, but in specific embodiments, it is on the order of minutes. In particular cases, the duration of exposure of the radiofrequency therapy for the individual is between 1-60, 1-50, 1-40, 1-30, 1-20, 1-10, 5-60, 5-50, 5-40, 5-30, 5-20, 10-45, 10-30, 10-20, 20-40, 20-30, 30-60, 45-60, 5-10, 5-9, 5-8, 5-7, 5-6, 6-10, 6-10, 6-8, 6-7, 7-10, 7-9, 7-8, 8-10, 8-9, or 9-10 minutes. The duration of the exposure of the RF therapy for the individual may be 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60 minutes in length. In cases wherein multiple exposures are provided to an individual, the different exposures may or may not be for the same duration in time.

In specific embodiments, the non-invasive radiofrequency therapy and one or more immunotherapies are provided to the individual once a day or more than once a day. The non-invasive radiofrequency therapy and one or more immunotherapies may be provided to the individual once a week or more than once a week. The non-invasive radiofrequency therapy and one or more immunotherapies may be provided to the individual over the course of weeks or months. In cases wherein there are multiple deliveries of the one or more immunotherapies as part of the combination with non-invasive radiofrequency therapy, one or more of the multiple deliveries may be of an immunotherapy that is not the immunotherapy that was initially employed in the combination therapy.

### II. Immunotherapy Embodiments

The methods and compositions of the disclosure include combination therapies that employ one or more immunotherapies. The immunotherapies may be of any kind, but in specific cases they include one or more immune checkpoint inhibitors, INOS inhibitors, antibodies, antibody fragments, and/or immune cells (such as engineered T cells, including CAR T cells, NK cells, or NKT cells, etc.). Additional immunotherapeutic approaches that may be used in combination with the method include at least one or more of the following: therapeutic cancer vaccines targeting various classes of tumor antigens; innate immune stimulation by targeting pattern recognition receptors such as agonists of Toll-like receptors 9, 7, 8, 4, or other innate immune sensing molecules; adoptive cellular immunotherapy such as infusion of cultured tumor infiltrating lymphocytes (TIL) or chimeric antigen receptor (CAR) T cells or NK-T cells, or natural killer (NK) cells. The immunotherapy may comprise combinations of cyclophosphamide, an iNOS inhibitor, cisplatin, and/or radiation.

Examples of immune checkpoint inhibitors include CTLA-4, PD1, or PD-L1. In alternative cases, the immunotherapy is not an immune checkpoint inhibitor. In specific cases, the immunotherapy is an INOS inhibitor, such as LNIL, L-NMMA 1400 W dihydrochloride, AR-C 102222, AMT hydrochloride, S-Isopropylisothiourea hydrobromide, Aminoguanidine hydrochloride, BYK 191023 dihydrochloride, EIT hydrobromide, (S)-Methylisothiourea sulfate or a combination thereof. The immunotherapy may be cyclosphosphamide, in certain cases.

Pharmaceutical compositions of the present disclosure comprise an effective amount of one or more immunotherapies dissolved or dispersed in a pharmaceutically acceptable carrier. The phrases “pharmaceutical” and “pharmacologically acceptable” and used interchangeably herein refer to molecular entities and compositions that do not produce an adverse, allergic or other untoward reaction when administered to an animal, such as, for example, a human, as appropriate, and do not interfere with the therapeutic methods of the disclosure. The preparation of an pharmaceutical composition that contains at least one immunotherapy or additional active ingredient will be known to those of skill in the art in light of the present disclosure, as exemplified by Remington: The Science and Practice of Pharmacy, 21st Ed. Lippincott Williams and Wilkins, 2005, incorporated herein by reference. Moreover, for animal (e.g., human) administration, it will be understood that preparations should meet sterility, pyrogenicity, general safety and purity standards as required by FDA Office of Biological Standards.

As used herein, “pharmaceutically acceptable carrier” includes any and all solvents, dispersion media, coatings, surfactants, antioxidants, preservatives (e.g., antibacterial agents, antifungal agents), isotonic agents, absorption delaying agents, salts, preservatives, drugs, drug stabilizers, gels, binders, excipients, disintegration agents, lubricants, sweetening agents, flavoring agents, dyes, such like materials and combinations thereof, as would be known to one of ordinary skill in the art (see, for example, Remington's Pharmaceutical Sciences, 18th Ed. Mack Printing Company, 1990, pp. 1289-1329, incorporated herein by reference). Except insofar as any conventional carrier is incompatible with the active ingredient, its use in the pharmaceutical compositions is contemplated.

The immunotherapy may comprise different types of carriers depending on whether it is to be administered in solid, liquid or aerosol form, and whether it needs to be sterile for such routes of administration, such as injection. The immunotherapy of the present disclosure can be administered intravenously, intradermally, transdermally, intrathecally, intraarterially, intraperitoneally, intranasally, intravaginally, intrarectally, topically, intramuscularly, subcutaneously, mucosally, intratumorally, orally, topically, locally, inhalation (e.g., aerosol inhalation), injection, infusion, continuous infusion, localized perfusion bathing target cells directly, via a catheter, via a lavage, in creams, in lipid compositions (e.g., liposomes), or by other method or any combination of the forgoing as would be known to one of ordinary skill in the art (see, for example, Remington's Pharmaceutical Sciences, 18th Ed. Mack Printing Company, 1990, incorporated herein by reference).

The immunotherapy may be formulated into a composition in a free base, neutral or salt form. Pharmaceutically acceptable salts, include the acid addition salts, e.g., those formed with the free amino groups of a proteinaceous composition, or which are formed with inorganic acids such as for example, hydrochloric or phosphoric acids, or such organic acids as acetic, oxalic, tartaric or mandelic acid. Salts formed with the free carboxyl groups can also be derived from inorganic bases such as for example, sodium, potassium, ammonium, calcium or ferric hydroxides; or such organic bases as isopropylamine, trimethylamine, histidine or procaine. Upon formulation, solutions will be administered in a manner compatible with the dosage formulation and in such amount as is therapeutically effective. The formulations are easily administered in a variety of dosage forms such as formulated for parenteral administrations such as injectable solutions, or aerosols for delivery to the lungs, or formulated for alimentary administrations such as drug release capsules and the like.

Further in accordance with the present disclosure, the composition of the present disclosure suitable for administration may be provided in a pharmaceutically acceptable carrier with or without an inert diluent. The carrier should be assimilable and includes liquid, semi-solid, i.e., pastes, or solid carriers. Except insofar as any conventional media, agent, diluent or carrier is detrimental to the recipient or to the therapeutic effectiveness of a composition contained therein, its use in practicing the methods of the present disclosure is appropriate. Examples of carriers or diluents include fats, oils, water, saline solutions, lipids, liposomes, resins, binders, fillers and the like, or combinations thereof. The composition may also comprise various antioxidants to retard oxidation of one or more component. Additionally, the prevention of the action of microorganisms can be brought about by preservatives such as various antibacterial and antifungal agents, including but not limited to parabens (e.g., methylparabens, propylparabens), chlorobutanol, phenol, sorbic acid, thimerosal or combinations thereof.

In accordance with the present disclosure, the composition is combined with the carrier in any convenient and practical manner, i.e., by solution, suspension, emulsification, admixture, encapsulation, absorption and the like. Such procedures are routine for those skilled in the art.

In a specific embodiment of the present disclosure, the composition is combined or mixed thoroughly with a semi-solid or solid carrier. The mixing can be carried out in any convenient manner such as grinding. Stabilizing agents can be also added in the mixing process in order to protect the composition from loss of therapeutic activity, i.e., denaturation in the stomach. Examples of stabilizers for use in an the composition include buffers, amino acids such as glycine and lysine, carbohydrates such as dextrose, mannose, galactose, fructose, lactose, sucrose, maltose, sorbitol, mannitol, etc.

In further embodiments, the present disclosure may include the use of a pharmaceutical lipid vehicle compositions that incorporates an immunotherapy, one or more lipids, and an aqueous solvent. As used herein, the term “lipid” will be defined to include any of a broad range of substances that is characteristically insoluble in water and extractable with an organic solvent. This broad class of compounds is well known to those of skill in the art, and as the term “lipid” is used herein, it is not limited to any particular structure. Examples include compounds which contain long-chain aliphatic hydrocarbons and their derivatives. A lipid may be naturally occurring or synthetic (i.e., designed or produced by man). However, a lipid is usually a biological substance. Biological lipids are well known in the art, and include for example, neutral fats, phospholipids, phosphoglycerides, steroids, terpenes, lysolipids, glycosphingolipids, glycolipids, sulphatides, lipids with ether and ester-linked fatty acids and polymerizable lipids, and combinations thereof. Of course, compounds other than those specifically described herein that are understood by one of skill in the art as lipids are also encompassed by the compositions and methods of the present disclosure.

One of ordinary skill in the art would be familiar with the range of techniques that can be employed for dispersing a composition in a lipid vehicle. For example, the immunotherapy may be dispersed in a solution containing a lipid, dissolved with a lipid, emulsified with a lipid, mixed with a lipid, combined with a lipid, covalently bonded to a lipid, contained as a suspension in a lipid, contained or complexed with a micelle or liposome, or otherwise associated with a lipid or lipid structure by any means known to those of ordinary skill in the art. The dispersion may or may not result in the formation of liposomes.

The actual dosage amount of a composition of the present disclosure administered to the subject can be determined by physical and physiological factors such as body weight, severity of condition, the type of disease being treated, previous or concurrent therapeutic interventions, idiopathy of the subject and on the route of administration. Depending upon the dosage and the route of administration, the number of administrations of a preferred dosage and/or an effective amount may vary according to the response of the subject. The practitioner responsible for administration will, in any event, determine the concentration of active ingredient(s) in a composition and appropriate dose(s) for the individual subject.

In certain embodiments, pharmaceutical compositions may comprise, for example, at least about 0.1% (by weight) of an active compound. In other embodiments, the active compound may comprise between about 2% to about 75% of the weight of the unit, or between about 25% to about 60%, for example, and any range derivable therein. Naturally, the amount of active compound(s) in each therapeutically useful composition may be prepared in such a way that a suitable dosage will be obtained in any given unit dose of the compound. Factors such as solubility, bioavailability, biological half-life, route of administration, product shelf life, as well as other pharmacological considerations will be contemplated by one skilled in the art of preparing such pharmaceutical formulations, and as such, a variety of dosages and treatment regimens may be desirable.

In other non-limiting examples, a dose may also comprise from about 1 microgram/kg/body weight, about 5 microgram/kg/body weight, about 10 microgram/kg/body weight, about 50 microgram/kg/body weight, about 100 microgram/kg/body weight, about 200 microgram/kg/body weight, about 350 microgram/kg/body weight, about 500 microgram/kg/body weight, about 1 milligram/kg/body weight, about 5 milligram/kg/body weight, about 10 milligram/kg/body weight, about 50 milligram/kg/body weight, about 100 milligram/kg/body weight, about 200 milligram/kg/body weight, about 350 milligram/kg/body weight, about 500 milligram/kg/body weight, to about 1000 mg/kg/body weight or more per administration of the active agent, e.g., an immunotherapy according to the present disclosure, and any range derivable therein. In non-limiting examples of a derivable range from the numbers listed herein, a range of about 5 mg/kg/body weight to about 100 mg/kg/body weight, about 5 microgram/kg/body weight to about 500 milligram/kg/body weight, etc., of the active agent can be administered, based on the numbers described above.

Alimentary Compositions and Formulations

In particular embodiments of the present disclosure, the immunotherapy is formulated to be administered via an alimentary route. Alimentary routes include all possible routes of administration in which the composition is in direct contact with the alimentary tract. Specifically, the pharmaceutical compositions disclosed herein may be administered orally, buccally, rectally, or sublingually. As such, these compositions may be formulated with an inert diluent or with an assimilable edible carrier, or they may be enclosed in hard- or soft-shell gelatin capsule, or they may be compressed into tablets, or they may be incorporated directly with the food of the diet.

In certain embodiments, the active compounds may be incorporated with excipients and used in the form of ingestible tablets, buccal tables, troches, capsules, elixirs, suspensions, syrups, wafers, and the like (Mathiowitz et al., 1997; Hwang et al., 1998; U.S. Pat. Nos. 5,641,515; 5,580,579 and 5,792, 451, each specifically incorporated herein by reference in its entirety). The tablets, troches, pills, capsules and the like may also contain the following: a binder, such as, for example, gum tragacanth, acacia, cornstarch, gelatin or combinations thereof; an excipient, such as, for example, dicalcium phosphate, mannitol, lactose, starch, magnesium stearate, sodium saccharine, cellulose, magnesium carbonate or combinations thereof; a disintegrating agent, such as, for example, corn starch, potato starch, alginic acid or combinations thereof; a lubricant, such as, for example, magnesium stearate; a sweetening agent, such as, for example, sucrose, lactose, saccharin or combinations thereof; a flavoring agent, such as, for example peppermint, oil of wintergreen, cherry flavoring, orange flavoring, etc. When the dosage unit form is a capsule, it may contain, in addition to materials of the above type, a liquid carrier. Various other materials may be present as coatings or to otherwise modify the physical form of the dosage unit. For instance, tablets, pills, or capsules may be coated with shellac, sugar, or both. When the dosage form is a capsule, it may contain, in addition to materials of the above type, carriers such as a liquid carrier. Gelatin capsules, tablets, or pills may be enterically coated. Enteric coatings prevent denaturation of the composition in the stomach or upper bowel where the pH is acidic. See, e.g., U.S. Pat. No. 5,629,001. Upon reaching the small intestines, the basic pH therein dissolves the coating and permits the composition to be released and absorbed by specialized cells, e.g., epithelial enterocytes and Peyer's patch M cells. A syrup of elixir may contain the active compound sucrose as a sweetening agent methyl and propylparabens as preservatives, a dye and flavoring, such as cherry or orange flavor. Of course, any material used in preparing any dosage unit form should be pharmaceutically pure and substantially non-toxic in the amounts employed. In addition, the active compounds may be incorporated into sustained-release preparation and formulations.

For oral administration, the immunotherapy compositions of the present disclosure may alternatively be incorporated with one or more excipients in the form of a mouthwash, dentifrice, buccal tablet, oral spray, or sublingual orally-administered formulation. For example, a mouthwash may be prepared incorporating the active ingredient in the required amount in an appropriate solvent, such as a sodium borate solution (Dobell's Solution). Alternatively, the active ingredient may be incorporated into an oral solution such as one containing sodium borate, glycerin and potassium bicarbonate, or dispersed in a dentifrice, or added in a therapeutically-effective amount to a composition that may include water, binders, abrasives, flavoring agents, foaming agents, and humectants. Alternatively, the compositions may be fashioned into a tablet or solution form that may be placed under the tongue or otherwise dissolved in the mouth.

Additional formulations which are suitable for other modes of alimentary administration include suppositories. Suppositories are solid dosage forms of various weights and shapes, usually medicated, for insertion into the rectum. After insertion, suppositories soften, melt or dissolve in the cavity fluids. In general, for suppositories, traditional carriers may include, for example, polyalkylene glycols, triglycerides or combinations thereof. In certain embodiments, suppositories may be formed from mixtures containing, for example, the active ingredient in the range of about 0.5% to about 10% (by weight), and preferably about 1% to about 2% (by weight).

Parenteral Compositions and Formulations

In further embodiments, the immunotherapy may be administered via a parenteral route. As used herein, the term “parenteral” includes routes that bypass the alimentary tract. Specifically, the pharmaceutical compositions disclosed herein may be administered for example, but not limited to intravenously, intradermally, intramuscularly, intraarterially, intrathecally, subcutaneous, or intraperitoneally U.S. Pat. Nos. 6,7537,514, 6,613,308, 5,466,468, 5,543,158; 5,641,515; and 5,399,363 (each specifically incorporated herein by reference in its entirety).

Solutions of the active compounds as free base or pharmacologically acceptable salts may be prepared in water suitably mixed with a surfactant, such as hydroxypropylcellulose. Dispersions may also be prepared in glycerol, liquid polyethylene glycols, and mixtures thereof and in oils. Under ordinary conditions of storage and use, these preparations contain a preservative to prevent the growth of microorganisms. The pharmaceutical forms suitable for injectable use include sterile aqueous solutions or dispersions and sterile powders for the extemporaneous preparation of sterile injectable solutions or dispersions (see, e.g., U.S. Pat. No. 5,466,468, specifically incorporated herein by reference in its entirety). In all cases the form must be sterile and must be fluid to the extent that easy injectability exists. It must be stable under the conditions of manufacture and storage and must be preserved against the contaminating action of microorganisms, such as bacteria and fungi. The carrier can be a solvent or dispersion medium containing, for example, water, ethanol, polyol (i.e., glycerol, propylene glycol, and liquid polyethylene glycol, and the like), suitable mixtures thereof, and/or vegetable oils. Proper fluidity may be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

For parenteral administration in an aqueous solution, for example, the solution should be suitably buffered if necessary and the liquid diluent first rendered isotonic with sufficient saline or glucose. These particular aqueous solutions are especially suitable for intravenous, intramuscular, subcutaneous, and intraperitoneal administration. In this connection, sterile aqueous media that can be employed will be known to those of skill in the art in light of the present disclosure. For example, one dosage may be dissolved in isotonic NaCl solution and either added hypodermoclysis fluid or injected at the proposed site of infusion, (see for example, “Remington's Pharmaceutical Sciences” 15th Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

Sterile injectable solutions are prepared by incorporating the active compounds in the required amount in the appropriate solvent with various of the other ingredients enumerated above, as required, followed by filtered sterilization. Generally, dispersions are prepared by incorporating the various sterilized active ingredients into a sterile vehicle which contains the basic dispersion medium and the required other ingredients from those enumerated above. In the case of sterile powders for the preparation of sterile injectable solutions, the preferred methods of preparation are vacuum-drying and freeze-drying techniques which yield a powder of the active ingredient plus any additional desired ingredient from a previously sterile-filtered solution thereof. A powdered composition is combined with a liquid carrier such as, e.g., water or a saline solution, with or without a stabilizing agent.

Miscellaneous Pharmaceutical Compositions and Formulations

In other preferred embodiments of the disclosure, the active compound immunotherapy may be formulated for administration via various miscellaneous routes, for example, topical (i.e., transdermal) administration, mucosal administration (intranasal, vaginal, etc.) and/or inhalation.

Pharmaceutical compositions for topical administration may include the active compound formulated for a medicated application such as an ointment, paste, cream or powder. Ointments include all oleaginous, adsorption, emulsion and water-solubly based compositions for topical application, while creams and lotions are those compositions that include an emulsion base only. Topically administered medications may contain a penetration enhancer to facilitate adsorption of the active ingredients through the skin. Suitable penetration enhancers include glycerin, alcohols, alkyl methyl sulfoxides, pyrrolidones and luarocapram. Possible bases for compositions for topical application include polyethylene glycol, lanolin, cold cream and petrolatum as well as any other suitable absorption, emulsion or water-soluble ointment base. Topical preparations may also include emulsifiers, gelling agents, and antimicrobial preservatives as necessary to preserve the active ingredient and provide for a homogenous mixture. Transdermal administration of the present disclosure may also comprise the use of a “patch”. For example, the patch may supply one or more active substances at a predetermined rate and in a continuous manner over a fixed period of time.

In certain embodiments, the pharmaceutical immunotherapy compositions may be delivered by eye drops, intranasal sprays, inhalation, and/or other aerosol delivery vehicles. Methods for delivering compositions directly to the lungs via nasal aerosol sprays has been described e.g., in U.S. Pat. Nos. 5,756,353 and 5,804,212 (each specifically incorporated herein by reference in its entirety). Likewise, the delivery of drugs using intranasal microparticle resins (see, e.g., Takenaga et al., 1998) and lysophosphatidyl-glycerol compounds (see, e.g., U.S. Pat. No. 5,725,871, specifically incorporated herein by reference in its entirety) are also well-known in the pharmaceutical arts. Likewise, transmucosal drug delivery in the form of a polytetrafluoroetheylene support matrix is described in, e.g., U.S. Pat. No. 5,780,045 (specifically incorporated herein by reference in its entirety).

The term aerosol refers to a colloidal system of finely divided solid of liquid particles dispersed in a liquefied or pressurized gas propellant. The typical aerosol of the present disclosure for inhalation will consist of a suspension of active ingredients in liquid propellant or a mixture of liquid propellant and a suitable solvent. Suitable propellants include hydrocarbons and hydrocarbon ethers. Suitable containers will vary according to the pressure requirements of the propellant. Administration of the aerosol will vary according to subject's age, weight and the severity and response of the symptoms.

Kits Comprising the Immunotherapy

Any of the immunotherapy compositions described herein may be part of a kit. The kits may comprise a suitably aliquoted immunotherapy of the present disclosure, and the component(s) of the kits may be packaged either in aqueous media or in lyophilized form. The container of the kits will generally include at least one vial, test tube, flask, bottle, syringe or other container, into which a component may be placed, and preferably, suitably aliquoted. Where there is more than one component in the kit, the kit also will generally contain a second, third or other additional container into which the additional component(s) may be separately placed. However, various combinations of components may be comprised in a vial. The kits of the present disclosure also will typically include container for holding the immunotherapy and any other reagent containers in close confinement for commercial sale.

When the components of the kit are provided in one and/or more liquid solutions, the liquid solution is an aqueous solution, with a sterile aqueous solution being contemplated. The compositions may also be formulated into a syringeable composition. In which case, the container may itself be a syringe, pipette, and/or other such like apparatus, from which the formulation may be applied to a particular area of the body, injected into an individual, and/or even applied to and/or mixed with the other components of the kit. However, the component(s) of the kit may be provided as dried powder(s). When reagents and/or components are provided as a dry powder, the powder can be reconstituted by the addition of a suitable solvent. It is envisioned that the solvent may also be provided in another container.

### EXAMPLES

The following examples are included to demonstrate preferred embodiments of the invention. It should be appreciated by those of skill in the art that the techniques disclosed in the examples that follow represent techniques discovered by the inventors to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1

### Non-Invasive Radiofrequency Field Treatment of 4T1 Breast Tumors Induces T-Cell Specific Inflammatory Response

Radiofrequency field treatment (RFT) has long been investigated as a potential therapy modality for solid tumors. Previous work by our lab and others has demonstrated that non-invasive RFT can increase tumoral blood perfusion, enhance localization of intravenously delivered drugs, and promote a variety of hyperthermic effects in solid tumors. More recently, we have discovered that RFT can modulate the intra-tumoral immune microenvironment. Using a 4T1 murine breast cancer model in immune competent Balb/c mice we were able to show significant increases in tumor size following RFT compared to mice that received similar systemic heating. Of interest, this increase in tumor size was absent in athymic nude Balb/c mice, which lack functional T-cells, leading us to infer that the RFT growth effect is T-cell specific. To investigate this further we performed an immune microenvironment study in which 4T1 tumor-bearing mice underwent a single dose of RFT followed by leukocyte subset analyses of tumor, spleen, blood, and the tumor draining lymph node at 24, 48, or 120 hours post-treatment. This demonstrated a significant increase in the infiltration of CD4+ T-cells into the tumor 24 hours post-RFT. By 48 hours post-RFT a number of immune checkpoint markers were unregulated and the enhanced T-cell infiltration returned to baseline levels, indicating that immunosuppressive mechanisms are likely a contributing cause of the rapid T-cell dynamic induced by RFT. Tumor growth curves from this time course microenvironment study further showed that the RFT growth effect is also transient; with an increasing tumor sizes noted up to 72 hour following treatment but then returning to similar sizes as untreated mice by day 5 post-RFT. Overall, these data demonstrate that non-invasive RFT has potential immunomodulatory effects in solid tumors and future plans will investigate combination with immunotherapeutic strategies such as immune checkpoint inhibitors.

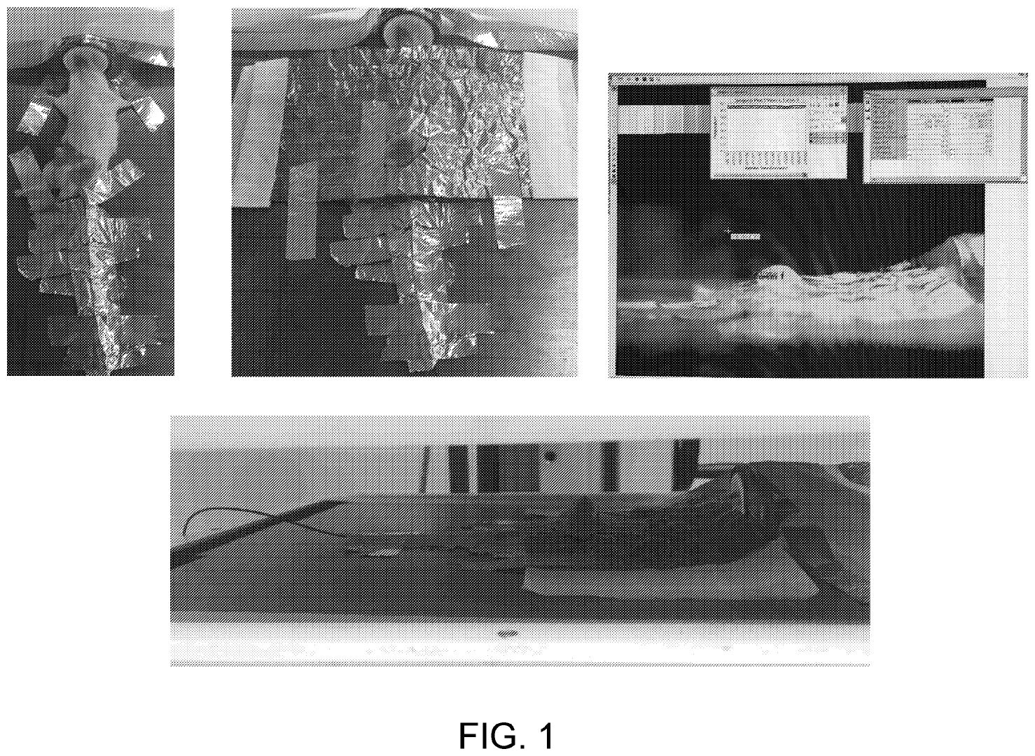

FIG. 1 shows one example of a set-up for how each mouse is RF-treated and monitored throughout the therapy. Image of top right shows IR-camera monitoring of skin temperature, which is one method for determining a delivered RF dose. Thermal dose could also be measured by non-reactive thermocouples placed into malignant and normal tissues, but this would be more invasive. Real-time magnetic resonance thermography can also be applied to measure thermal changes within tumors and adjacent nonmalignant tissues

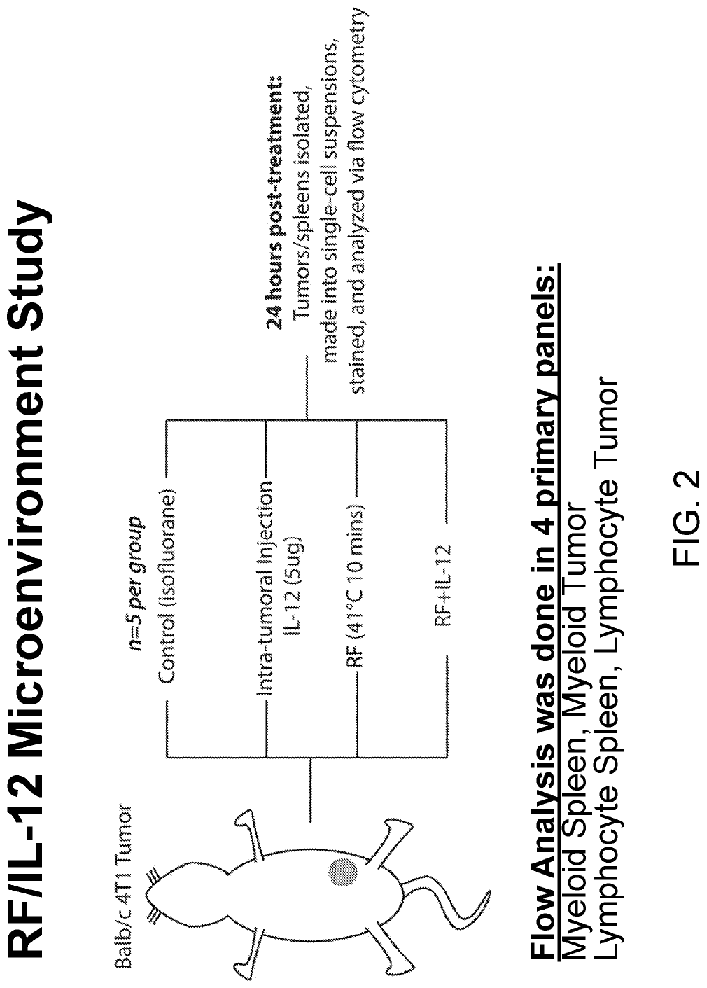

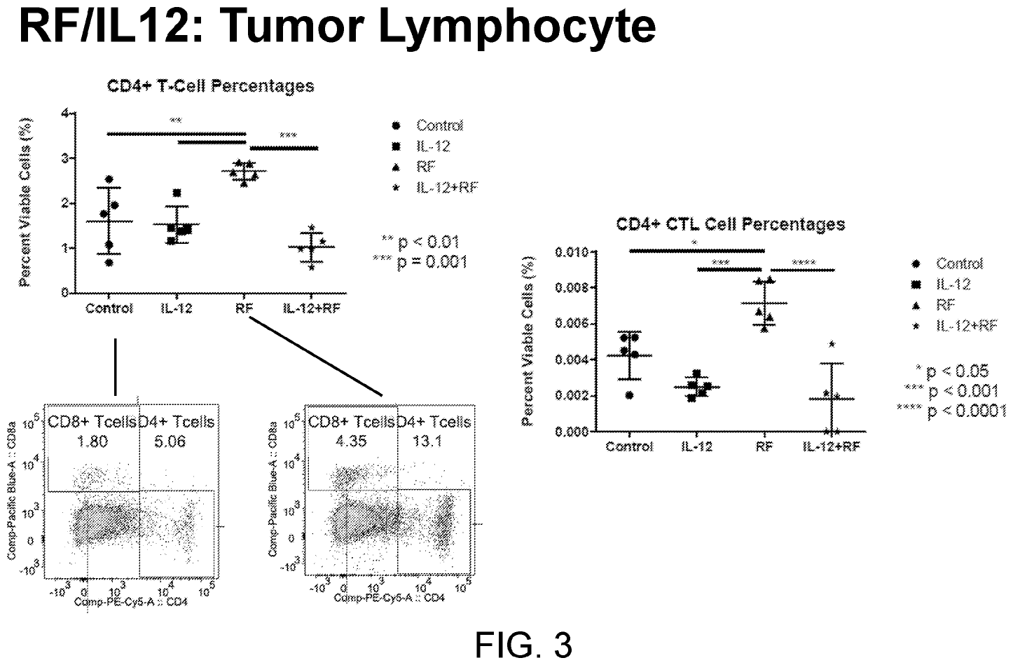

FIG. 2 shows an experimental design for one example of a microenvironment experiment. RF-treated mice were heated to 41° C. (as monitored via IR camera) and held at that temperature for 10 minutes. As a positive control, this experiment also included IL-12 intratumoral injections, and IL-12 is known to induce significant intra-tumoral immune responses and systemic immune responses. 24 hours after the mice were treated their tumors and spleen were isolated and processed via flow cytometry in order to assess the changes in intra-tumoral immune microenvironment. FIG. 3 shows the results from the experiment of the microenvironment study of FIG. 2. These data show that RF promotes a significant increase in CD4+ T-cell infiltration to the tumor, and that those T-cells appear to be of a “cytotoxic variety”. In specific aspects, those T-cells express cytolytic granules, such as Granzyme B and Perforin, for example.

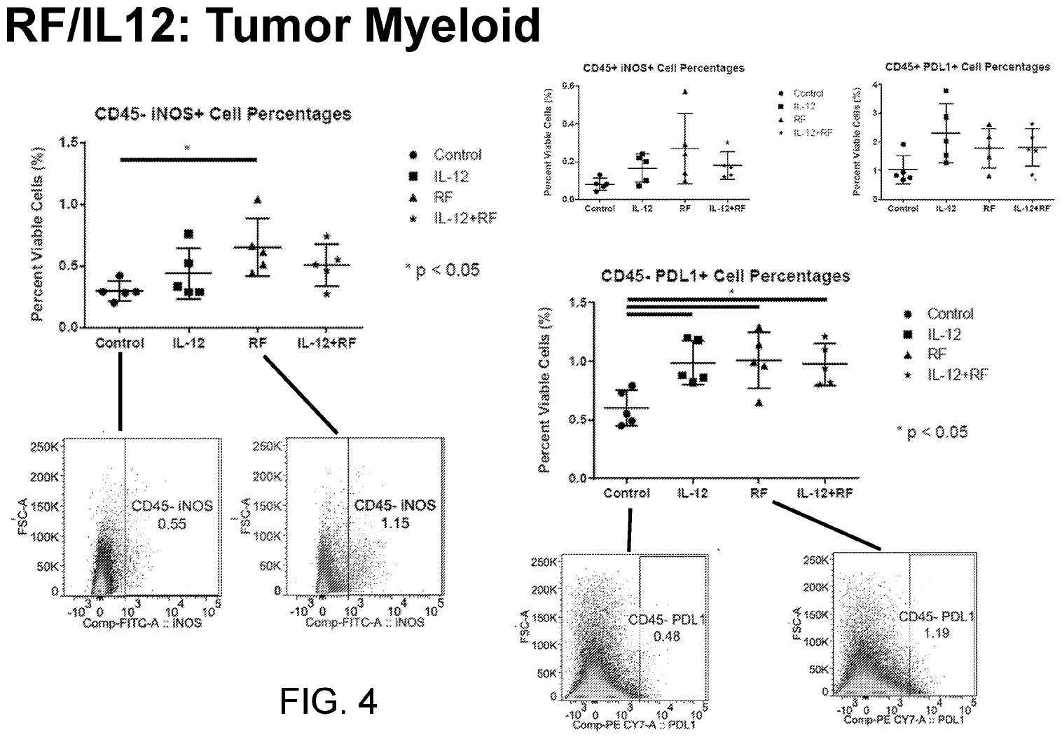

FIG. 4 demonstrates that RF upregulates both iNOS expression and PD-L1 expression (both immunosuppressive markers) in the tumor-residing cells (CD45 negative cells). This is evidence of an activated immune environment and yields evidence for combination strategies (i.e. iNOS inhibitors and immune checkpoint inhibitors).

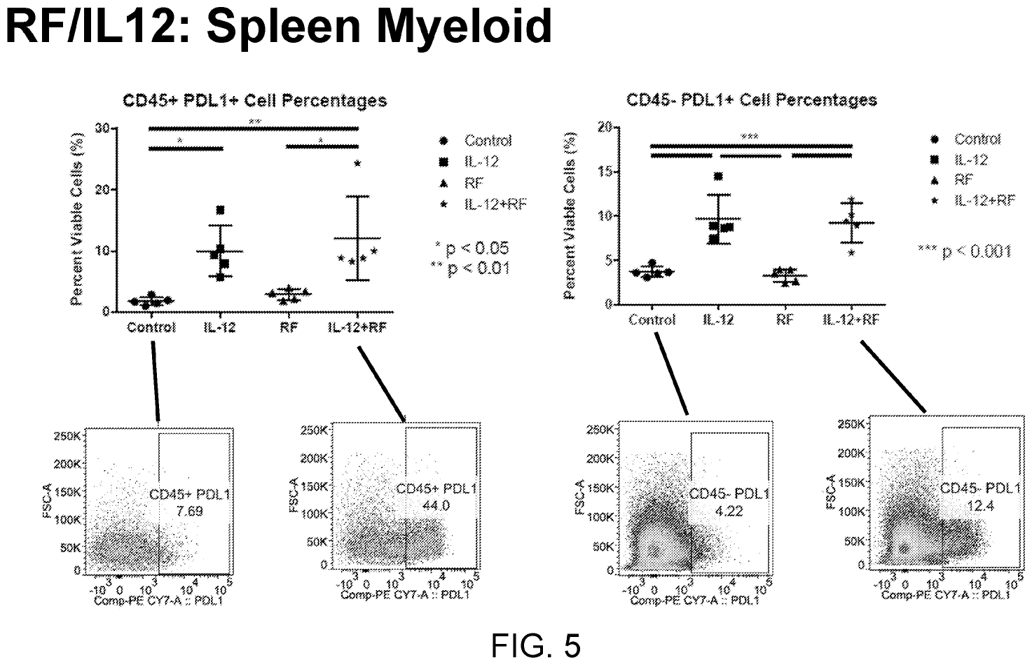

FIG. 5 shows data demonstrating that in the spleen of these mice there was no induced changes via RF (whereas IL-12 induced significant changes in PD-L1 expression). This is evidence that RF is acting locally to change the tumor immune microenvironment in the tumor, and in specific cases does not induce significant systemic immune effects.

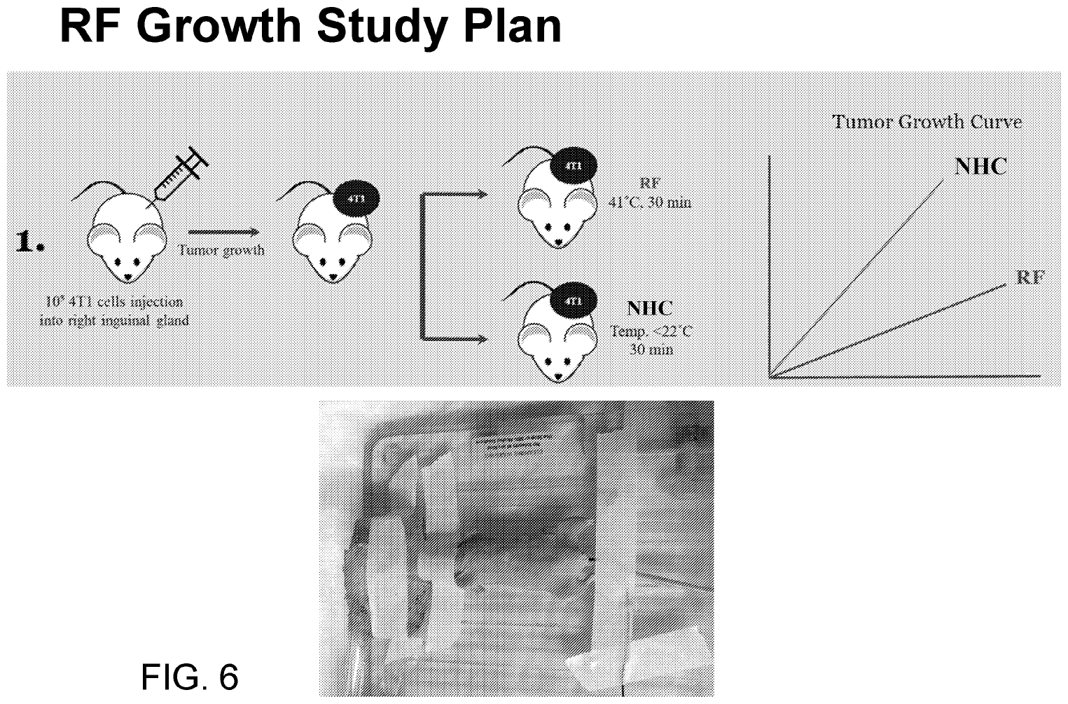

FIG. 6 demonstrates one experimental set-up for a tumor growth study. Balb/c mice with 4T1 (breast cancer) tumors were either RF-treated (41° C., 30 mins) or control treated through “No-Heat Control (NHC)” conditions. The manner in which the NHC mice were treated can be seen in the image to the left. These mice were placed on a heating platform and their systemic body temperature (as measure via a rectal probe) was maintained similar to what the RF treated mice systemic body temperature reached (RF mice systemic temperatures were also monitored via a rectal probe).

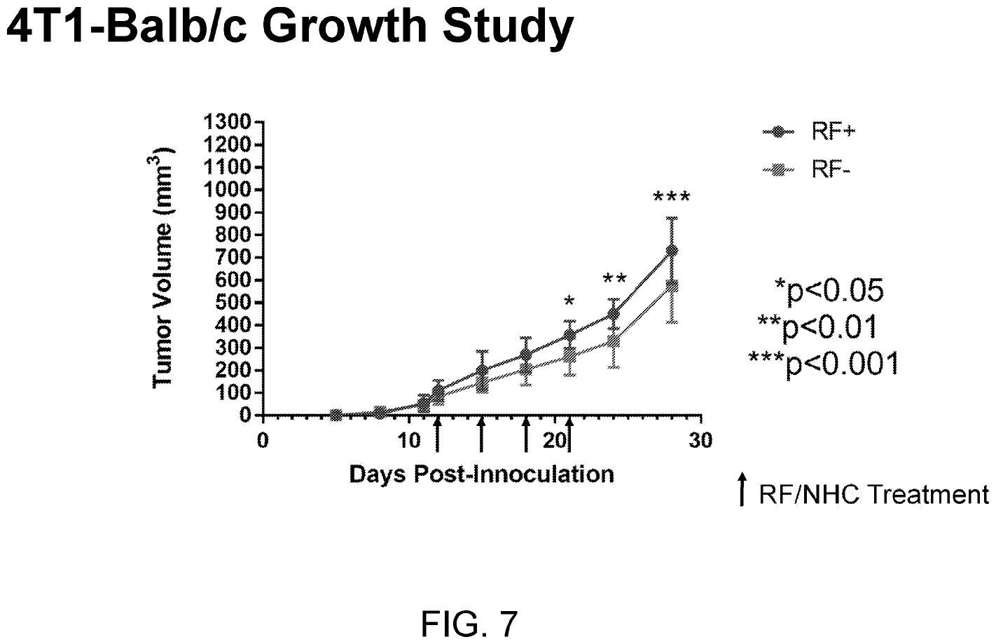

FIG. 7 shows the results from the tumor growth/survival study (n=10 per group). This shows that the RF-treated mice had a significant growth rate increase, and after 4 total RF treatments (as shown via the arrows in the plot) this growth difference was significant compared to the NHC-treated mice. This demonstrates that RF is inducing significant intratumoral inflammation within these tumors, thereby causing them to appear larger due to an enhanced infiltration of immune cell mediators.

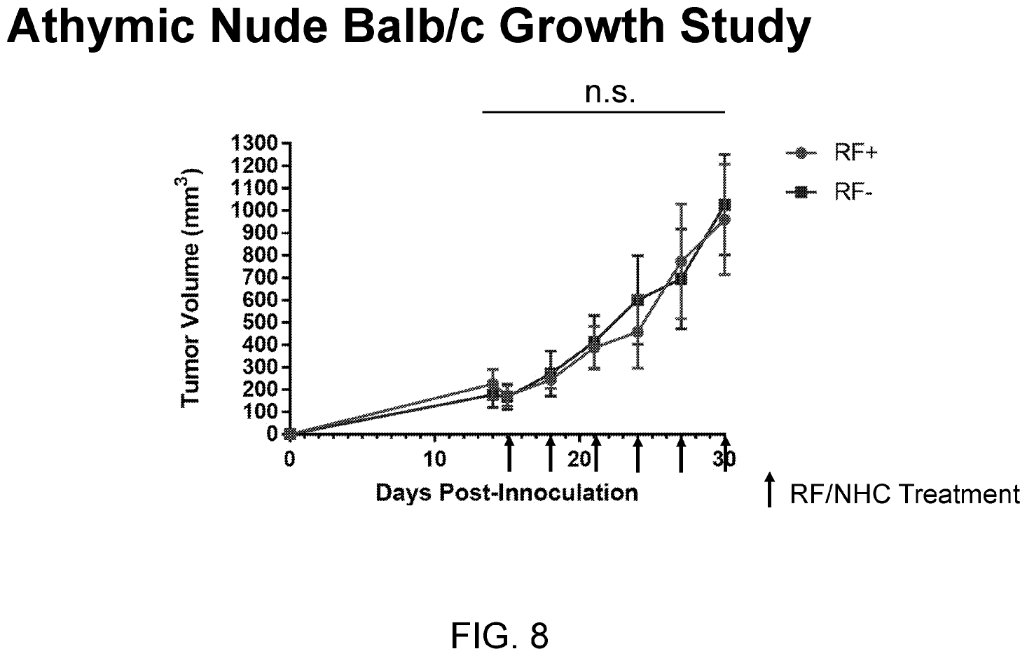

FIG. 8 provides a similar tumor growth/survival experiment performed in Athymic Nude Balb/c mice with 4T1 tumors. Athymic nude mice lack functional T-lymphocytes (because they lack a thymus, which is a critical organ needed for T-cell maturation). When these mice were similarly RF-treated, for a total of 6 RF doses (41° C., 30 mins), they showed no change in tumor size compared to NHC-mice. This is evidence that the significant intra-tumoral inflammation that was noted in the previous growth curve (FIG. 7), is dependent on T-cells. This is further supported by the microenvironment findings that showed that tumors that received RF had a significant increase in CD4+ T-cell infiltration.

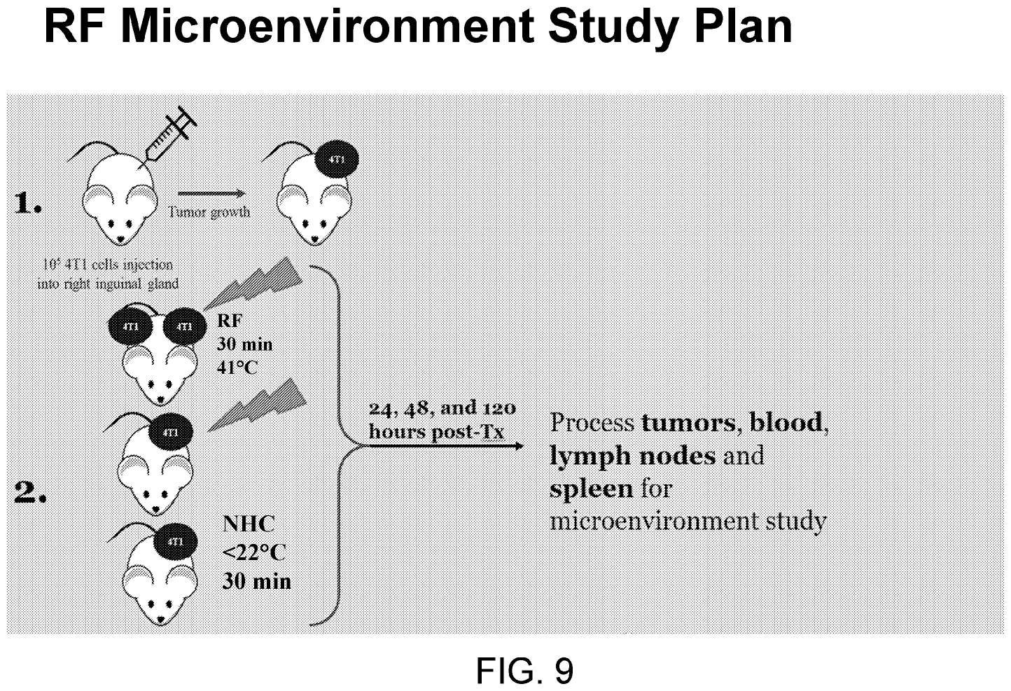

FIG. 9 provides a schematic that shows the experimental design for the 2nd microenvironment experiment. Again, Balb/c mice with 4T1 breast tumors were used. In this study, the inventors employed both singlet tumor mice that either did or did not receive RF, and they employed doublet tumor mice that only received RF treatment on one tumor (the other tumor was shielded using a copper blanket as seen in FIG. 1). Tumors, spleens, blood, and lymph nodes from these mice were then isolated at various times post-RF (24, 48, or 120 hours post-RF). These tissues were stained and processed via flow cytometry.

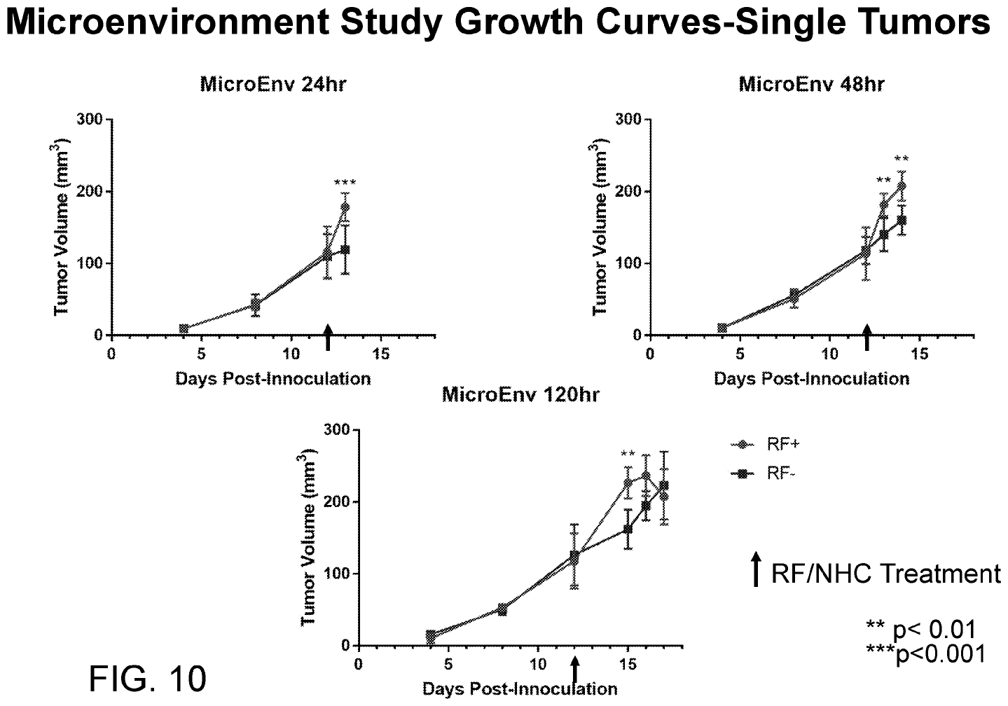

FIG. 10 plots show the tumor growth curves from the microenvironment treated mice between when they were RF-treated (day 12), and when they were sacked for tissue processing. These results show that by 24 hours post-RF there is a significant increase in the RF-treated mice tumor sizes, and this effect further increases at 48 hours post-RF. Finally, at least by 120 hours post-RF one can detect a return in size to the NHC treated mice, indicating that the RF inflammatory effects are transient.

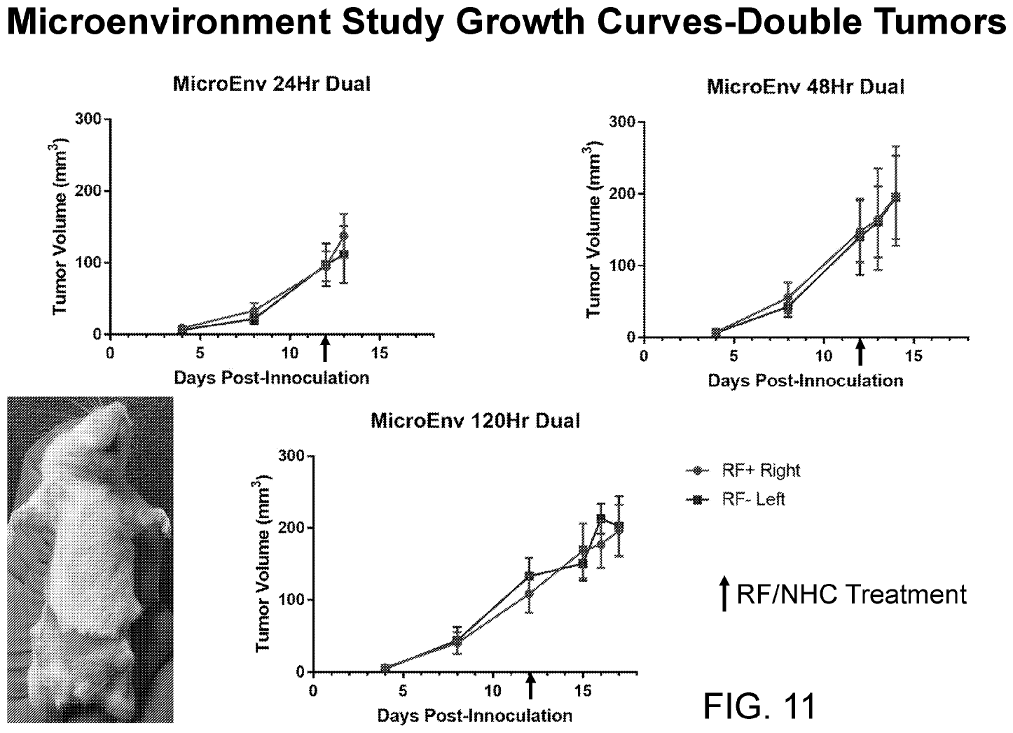

FIG. 11 plots show the growth curves for the dual tumor mice following a single RF-treatment (day 12) up until the day of tissue processing for the microenvironment study. There was no major difference in tumor size noted in these mice for any of the post-RF times. These plots thus are evidence that RF may induce an immune response that can promote inflammation of both a RF-treated primary tumor and distal tumors (i.e. metastases).

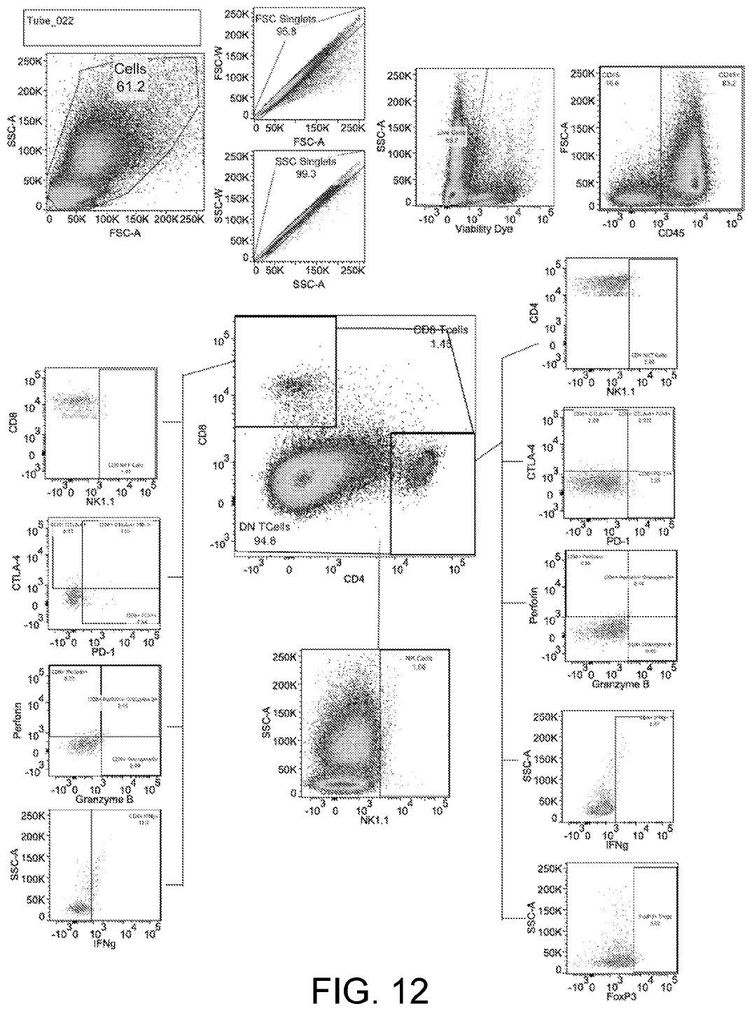

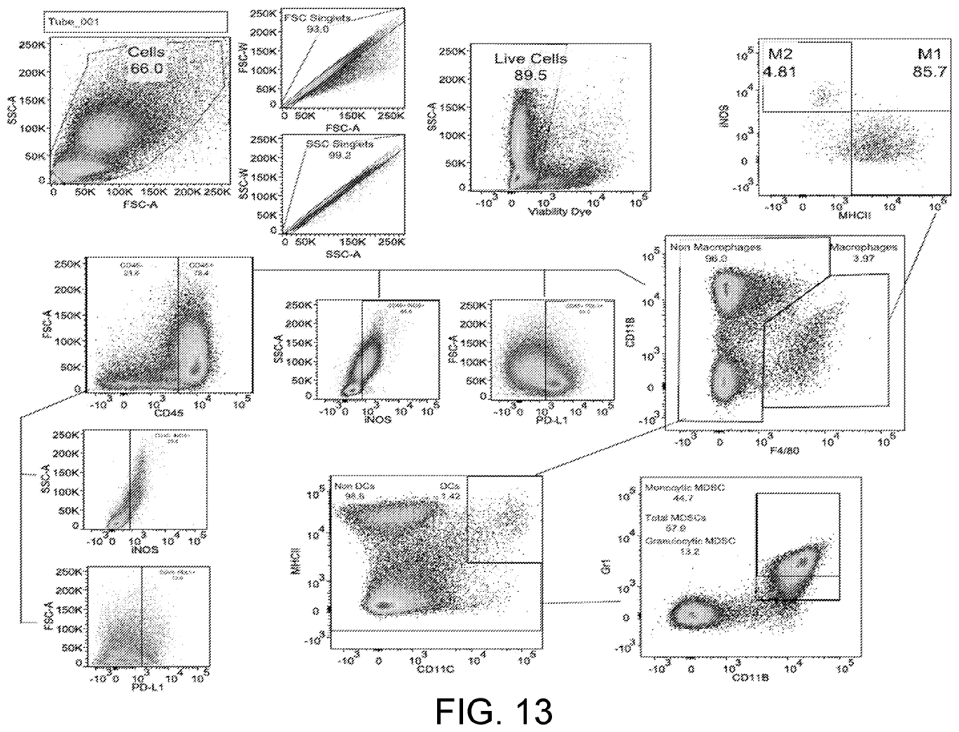

FIG. 12 shows a flow cytometry gating strategy for the lymphocyte staining panel that was used for the immune microenvironment studies, and FIG. 13 provides a schematic that shows the flow cytometry gating strategy for the myeloid staining panel that was used for the immune microenvironment studies.

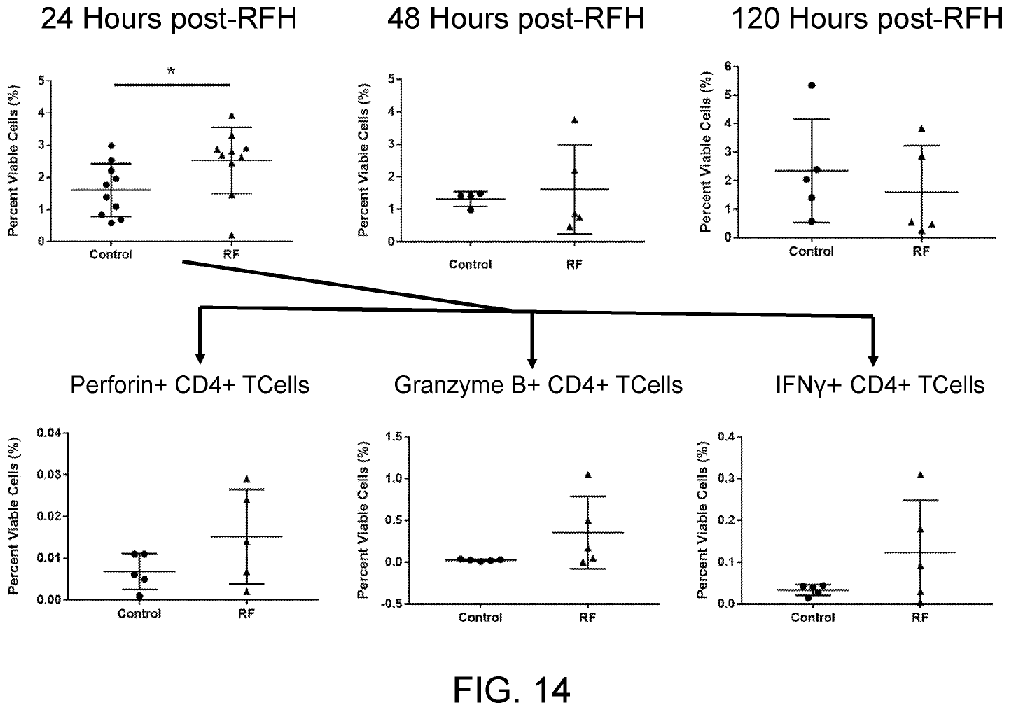

FIG. 14 provides the results from another microenvironment study. These plots show that in the tumor, there is a significant increase in CD4+ T-cells 24 hours after RF treatment, however that increase has diminished by 48 and 120 hours post-RF. In addition, those CD4+ T-cells at 24 hours post-RF express higher levels of various activation and cytotoxic markers such as Perforin, Granzyme B, and IFNγ. All of this directly replicates what was seen in the prior microenvironment study, thus demonstrating that this increase in cytotoxic/activated CD4+ T-cells is a robust effect of RF treatment.

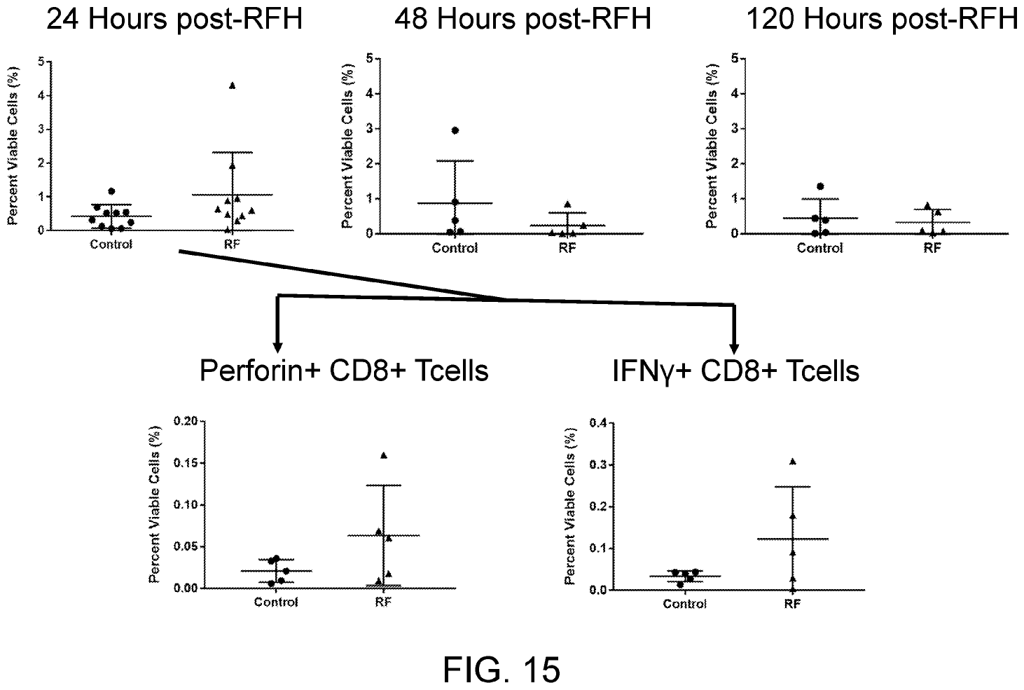

FIG. 15 plots show that there was no significant change in tumor infiltrating CD8+ T-cell at any of the monitored time points post-RF treatment. However, the CD8+ T-cells that are in the tumor at 24 hours post-treatment appear to express higher levels of the cytotoxic and activation markers Perforin and IFNγ, in the RF treated group. This suggests that the increased levels of CD4+ T-cells (also known as “helper” T-cells) in the tumor are activating more of the CD8+ T-cells in the tumor.

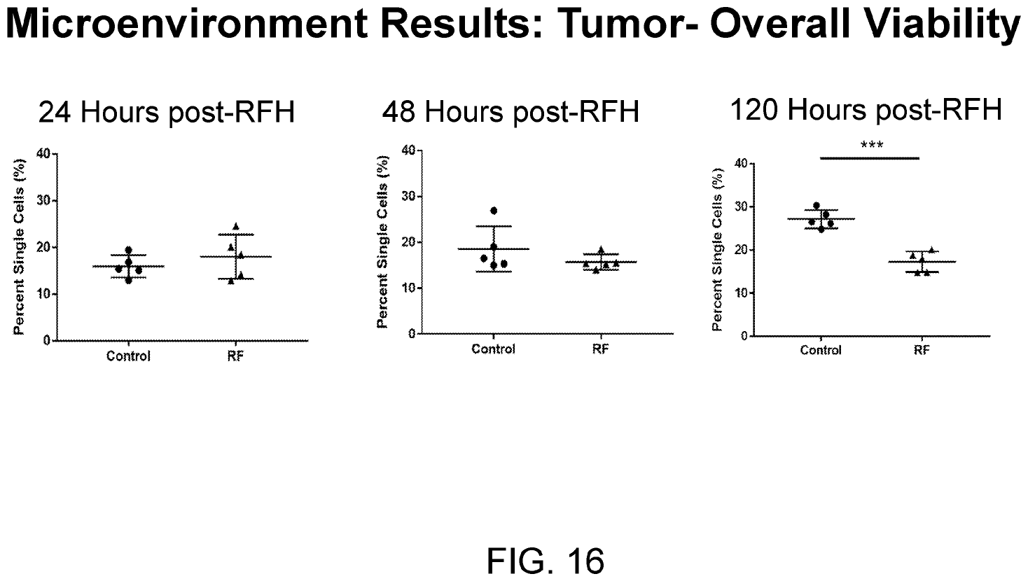

FIG. 16 plots show that by 120 hours post-RF, there is a significant decrease in tumor cell viability in the RF-treated group. This provides evidence that RF is inducing more of an immune response in these tumors, which is promoting more overall tumor death. In addition, this finding may simply indicate that because these tumors are larger, they possess a larger necrotic core.

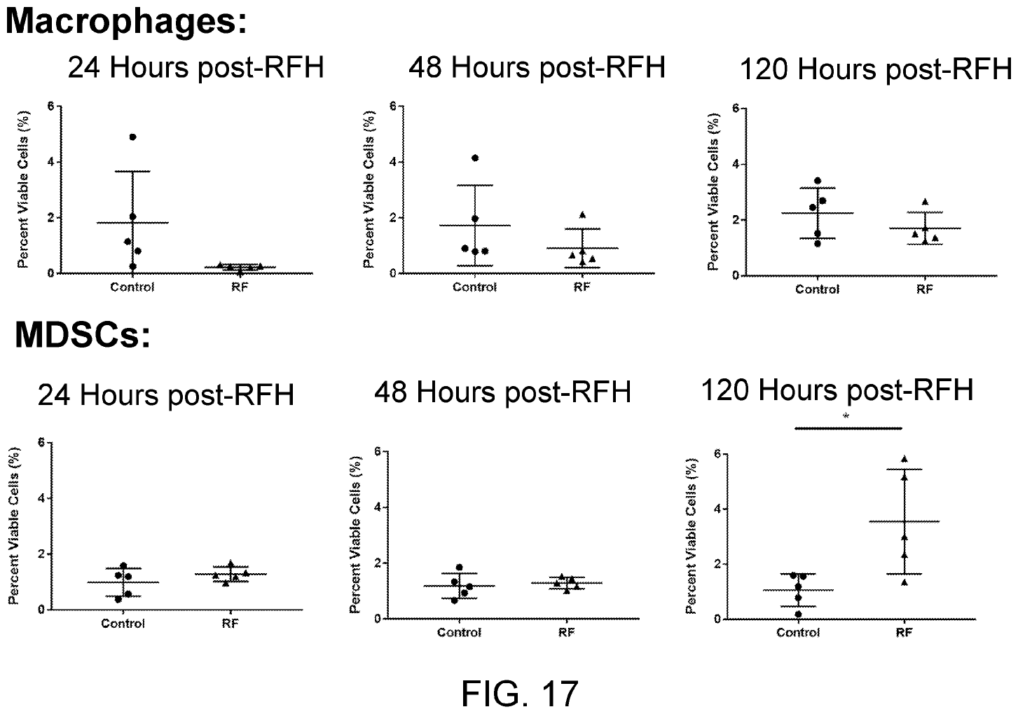

FIG. 17 plots show that RF treatment induces a significant decrease in intra-tumoral macrophages at 24 hours post-RF, which return to normal levels by 48 hours. The second row of plots indicate there is a significant increase in myeloid derived suppressor cells (MDSCs) by 120 hour post-RF. This decrease in macrophages could be because of enhanced macrophage activation by the enhanced CD4+ T-cells present in the tumor, in specific embodiments. The increase in MDSCs provide a useful combination strategy, whereby one could combine RF with a drug that will deactivate MDSCs (i.e., iNOS inhibitor) to enhance the long term effects of RF.

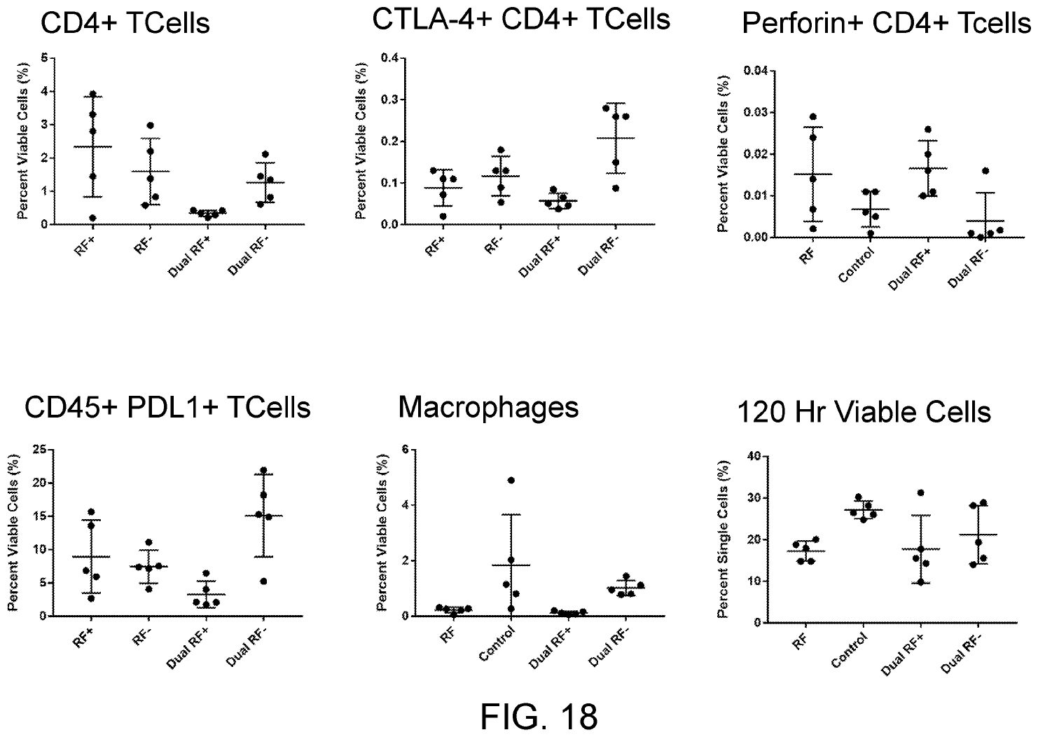

FIG. 18 provides plots that all show immune changes in the dual tumor mice. Of notice, all of these plots except for the bottom right plot are showing effects at 24 hours post-RF treatment. On each plot is shown the cellular percentages for the singlet tumor RF-treated mice, singlet tumor NHC mice, dual tumor mice RF-treated side, dual tumor mouse non-RF treated side (left to right). The non-RF treated side for the dual tumor mice showed enhanced immune activation. This included an enhancement in CD4+ T-cells, more CTLA-4 expression on those CD4+ T-cells (a marker of suppression that follows activation), and more PD-L1 expression on immune cells (another marker of suppression). Overall, this indicates that induction of an inflammatory immune response in the RF-treated tumor is promoting significant immune changes in the secondary tumor as well, thus suggesting that RF is inducing an abscopal effect, in specific embodiments.

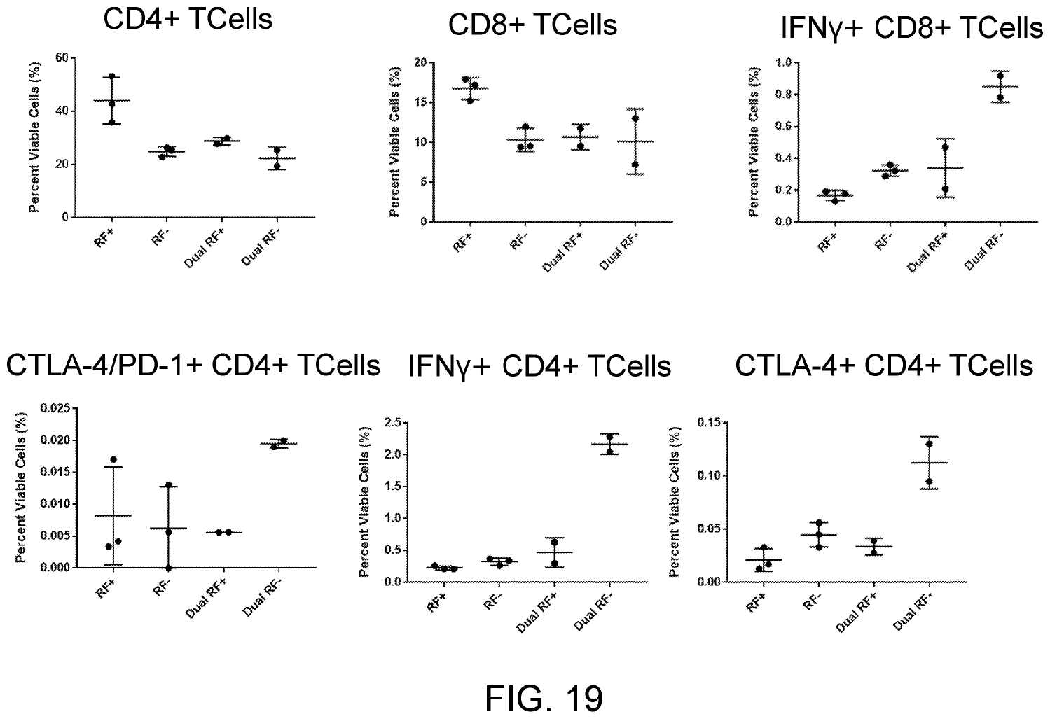

FIG. 19 plots show the immune changes in the tumor draining lymph nodes at 120 hours post-RF for both the singlet and dual tumor mice. One can detect an enhanced level of CD4 and CD8 T-cells in the singlet RF tumors, and there are higher levels of T-cell activation and suppression in the non-RF treated tumors in the dual tumor mice (i.e. higher levels of IFNγ, CTLA-4, and PD-1 in both CD4 and CD8 T-cells). This further provides evidence of the significant immune response induced by RF.

Samples from both the spleen and the blood did not show any significant changes for any of the various immune cell populations/features that were analyzed during the immune microenvironment studies, which reflected the results of the earlier microenvironment study in which there were no major differences in the splenic immune cell subsets. This is further evidence that the methods do not induce significant systemic immune changes. Instead, the RF effect remains very localized to the RF-treated tumor and distal tumor sites, in particular embodiments.

### Example 2

### Non-Invasive Radiofrequency Field Treatment of 4T1 Breast Tumors Induces T-Cell Dependent Inflammatory Response

Introduction—

Solid tumor cancers currently make up over 85% of new cancer cases and result in over 90% of all cancer-related deaths in the United States (Ferlay, et al., 2013). Despite promising advances in cancer oncology many solid tumors pose a major challenge, often resulting in the use of aggressive treatment schedules which expose patients to dangerous, potentially deadly, treatment regimens with considerable side-effects. This has led the field to seek alternative treatment options for solid tumor malignancies, and immunotherapy is currently a promising area of investigation. Immunotherapies hold substantial potential as a treatment platform for solid tumors, largely because they provide semi-selective targeting of cancer cells, the ability to attack both local and disseminated disease, and have features of immunologic memory which can recognize and eliminate instances of recurrence. These unique abilities of immunotherapy make it an increasingly attractive treatment strategy in solid tumor therapy.

Despite extensive efforts however, immunotherapy has currently shown minimal efficacy against solid tumor malignancies. Many groups propose that various tumor factors are primarily responsible for this lack of efficacy. The first of mention is the tumor immune microenvironment, a highly immunosuppressive environment composed of a variety of immunosuppressive cell types such as myeloid-derived suppressor cells (MDSC) and T-regulatory cells (Treg), which mitigate any potential effector immune responses infiltrating the tumor (Junttila, et al., 2013). Multiple groups have further suggested that tumors possess high intra-tumoral interstitial pressure and high levels of hypoxia due to inadequate blood vascularization which significantly hinders various cells of the immune system from reaching the internal recesses of the tumor (Huang, et al., 2013).

Non-invasive radiofrequency field treatment (RFT) has been previously investigated by groups as a potential therapy in both pancreatic ductal adenocarcinoma (PDAC) and hepatocellular carcinoma (HCC) (Koshkina, et al., 2014; Glazer, et al., 2010; Raoof, et al., 2013). Further characterization of the effects of RFT in in vitro systems suggests that RFT induces changes in cell-cell adhesion, elasticity, and morphology, which could majorly change the physical characteristics of the tumor microenvironment (Ware, et al., 2015; Ware, et al., 2017). Single dose-RFT can enhance intra-tumoral blood flow and perfusion of intravenously delivered nanoparticle probes (Corr, et al., 2015; Lapin, et al., 2017). More recently an optimal consecutive treatment regimen has been developed and it was observed that it enhanced intravenously delivered fluorescent probes, suggesting its potential role in enhancing tumoral drug delivery (Ware, et al., 2017). Finally, RFT promoted a unique form of tumor hyperthermia, with drastic improvements in temperature differential (i.e. internal tumor temperature vs. systemic body temperature), uniform tumor heating, post-treatment intra-tumoral blood velocity, and overall safety compared to contact-convectively delivered hyperthermia treatment (Ware, et al., 2017). Thus, RFT proved to be a safe and optimal method for exposing the tumor to hyperthermic conditions.

Hyperthermia has long been a topic of interest in cancer with treatment history dating back to Hippocrates in ancient Greece (Bull, et al., 1962). Systemic hyperthermia in the febrile range (38° C.-42° C.) has been previously investigated for its chemo- and radiotherapy sensitizing properties, which improved clinical outcomes in a randomized clinical trial (Issels, et al., 2010). With recent technological developments a number of studies have investigated more localized hyperthermia effects including isolated limb, intraperitoneal, and thoracic cavity heating and observed promising enhancements in tumor treatment sensitivity (Tillerman, et al., 2009; Verwaal, et al., 2003). In addition to radio- and chemo-sensitizing properties, hyperthermia is also known to promote a variety of favorable intratumoral immunologic effects (Repasky, et al., 2013). Several potential treatment mechanisms have been suggested including the ability of febrile range hyperthermia to improve tumor oxygenation, a critical hurdle of immune attack of solid tumors, as hypoxic conditions are known to promote many immunosuppressive effects (Huang, et al., 2013; Jones, et al., 2004). In addition to re-oxygenation, numerous studies have suggested that mild hyperthermia can promote effector immune cell tumoral trafficking, with some reported cases showing a greater than 5-fold increase in cytotoxic T cells infiltrating the tumor microenvironment (Fisher, et al., 2011). Although the mechanism of this enhanced infiltration still remains somewhat unclear, prior data would suggest that the mild hyperthermia is able to drive various features which favor lymphocyte infiltration and attack of solid tumors; these features include increasing expression of vessel wall binding domains such as intercellular adhesion molecule-1 (ICAM-1) (Chen, et al., 2009) [Lefor et al. Surgery, 1994], promoting a more favorable intratumoral interstitial pressure (Leunig, et al., 1992), and driving the production of a number of pro-inflammatory cytokines and chemokines (i.e. CCR1, IL-1β, IL-6, IL-8, IL-10) which have major implications in T-cell activation and trafficking (Mikucki, et al., 2013; Evans, et al., 2001). Despite the vast amount of work done to investigate the immunologic effects of intratumoral hyperthermia, most studies rely on either systemic or convectively delivered hyperthermia. Based on prior data, RFT can provide more localized, uniform, and safe intratumoral hyperthermia; however, an investigation of the immunologic effects following febrile-range RFT hyperthermia remains unknown.

Therefore, the objective of this Example was to characterize the immunologic changes induced by RFT with a particular focus on intratumoral immune microenvironment changes. In certain embodiments RFT would induce significant intra-tumoral immune changes, for example through pro-inflammatory mechanisms. Using immune competent Balb/c mice bearing 4T1 breast tumors, both consecutive RFT dosing and single-dose time-course schedules were investigated to characterize the transient immunologic changes induced by RFT.

Examples of Materials and Methods

Ethic statement and general mice conditions—All experiments were performed with approval of the Institutional Animal Care and Use Committee (IACUC) of Baylor College of Medicine (No. AN-6445) and following established protocols. Female Balb/c or athymic nude Balb/c mice (Jackson Labs) were housed in standard temperature and lighting conditions with free access to food and water. RFT was performed under isoflurane anesthesia (0.7-2.5% isoflurane in medical air). During anesthesia, systemic mouse temperature was monitored using a rectally inserted fiber optic temperature probe and breathing frequency was maintained at approximately 1 Hz by adjusting isoflurane concentration and/or flow rate. After anesthesia and treatment mice were kept in a pre-warmed chamber until complete recovery.

Tumor model—4T1 cells were purchased from American Type Cell Culture (ATCC; Rockville, Md.) and were cultured in RPMI 1640 media supplemented with 10% fetal bovine serum (FBS) and 1% penicillin/streptomycin. Cells were cultured in a humidified atmosphere at 37° C. and 5% CO₂. 10⁵ 4 T1 breast cancer cells suspended in base medium were injected into the left inguinal gland (27 G needle, 50 μL injection volume) to initiate orthotropic 4T1 breast tumors in Balb/c mice (wildtype or athymic nude). Treatment initiated between 12-15 days of tumor development when tumors had reached 100-200 mm³ in volume. All tumor measurements were performed using calipers and volume was calculated using Equation 1, where W=tumor width, L=tumor length, and H=tumor height.

$$V = \frac{π}{6} * L * W * H \qquad ( 1 )$$

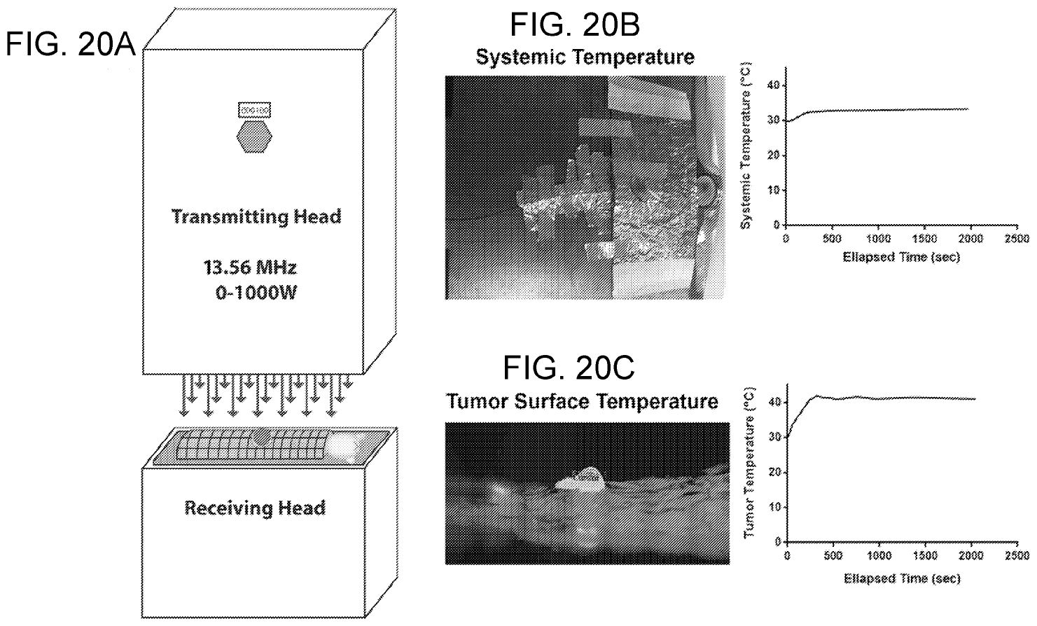

Radiofrequency field treatment (RFT)—For a single dose of RFT, anesthetized mice were grounded and shielded using copper tape to ensure localized RFT at the tumor site. Mice were then subjected to high intensity (˜90 kV/m) 13.56 MHz RF fields at various powers (0-1000 W) to administer a bi-phasic thermal dose that included a ‘ramp up’ phase from baseline tumor surface temperature to 41° C. and a second ‘plateau’ phase which maintained tumor surface temperature at 41° C. for 30 mins. Non-RF “control” mice were similarly anesthetized and placed on a heating pad to achieve similar levels of systemic heating without the addition of RFT. Systemic temperature and tumor surface temperature were measured using a rectally inserted fiber optic thermal probe and an infrared camera, respectively (for treatment set up see FIGS. 20A-20C). For both control and RFT mice, the skin on and around the tumor was shaved prior to treatment to allow for accurate tumor surface temperature assessment throughout treatment. All inoculated mice were randomized prior to treatment and the tumor measurements were performed with proper blinding. Before experiments were performed, the IR camera and fiber optic probes were calibrated using a water-bath (data not shown) to ensure accurate thermometry.

Multiple Dose RFT—

Consecutive doses of RFT or control treatment were performed in established 4T1 breast tumors in Balb/c mice (wildtype or athymic nude) as described above and in our previous work (Ware, et al., 2017). After tumors reached size requirements, treatment was initiated and repeated every 3 days for a total of 4-5 doses. Growth assessment was terminated between day 28-30 post-tumor inoculation.

Tissue Sectioning and Histology—

At termination, tumors were isolated, frozen in O.C.T (Tissue-Tek), and stored at −80° C. prior to histologic sectioning and staining. Frozen sections were cut at −20° C. at 6 μm thickness and picked up on positively charged slides. H&E stains were fixed in 95% ethyl alcohol for 2 minutes prior to standard H&E staining. Unstained slides were stored at −80° C. until further processing.

Ki67 Immunohistochemistry—

Slides were fixed in chilled acetone for 20 minute prior to staining. Heat-induced epitope retrieval was performed using Biocare Decloaking chamber and Rodent Decloaking solution at 125° C. for 30 minutes. Slides then underwent primary Ki67 antibody incubation for 30 minutes, MACH2 universal detection incubation for 30 minutes, and Betazoid DAB chromogen for 5 minutes, with thorough washing using Tris-buffered saline between each incubation. After the final rinse, slides were background stained using CAT hematoxylin for 5 minutes. Fully stained slides were thoroughly rinsed and dehydrated using an ethanol gradient series prior to coverslipping.

Microscopic Imaging of Histology—

All brightfield imaging was performed using a Nikon Eclipse TE2000-U microscope fitted with a Nikon digital sight DS-Fi1 camera. Image analysis was performed using ImageJ software (National Institutes of Health, USA).

Single Dose RFT Time-Course—

A single dose of RFT or control treatment was delivered at day 12 post-tumor inoculation and leukocyte subset analyses of tumor, spleen, blood, and tumor draining inguinal lymph node was performed at 24, 48, or 120 hrs after the single treatment.

Tissue Preparation—

At termination tumor, spleen, blood, and tumor draining inguinal lymph node were harvested. Tumor was manually chopped into pieces and digested in base media containing 1 mg/mL Collagenase I (Sigma) for 1 hr at 37° C. with gentle shaking throughout. At 1 hr, digestions were immediately ceased with the addition of media containing 2% FBS. Digested tumors, spleens, and lymph nodes were smashed through a 30 μm cell strainer to obtain single cell suspensions. Blood was collected via cardiac puncture and split between a tube containing EDTA to prevent clotting (for microenvironment analysis) and a free tube which was allowed to clot at room temperature (for serum cytokine collection). Single cell suspension of spleen and EDTA-containing blood underwent red blood cell lysis using LCK lysis buffer per manufacturer's instructions. Clotted blood from the free tube was centrifuged (2000×g, 10 mins) and supernatant serum was collected and stored at −80° C. prior to analysis.

Staining and Flow Cytometry—

Single cell suspensions for microenvironment analysis were stained for flow cytometric analysis using either a lymphocyte or myeloid staining panel. Antibodies for the lymphocyte panel are as follows; CD45 APC-eFluor780, CD4 PE-Cy5, CD8a eFluor450, NK1.1 AF700, PD-1 PerCP-eFluor710, CTLA-4 PE, IFNγ PE-eFluor610, Granzyme B eFluor660, Perforin FITC, and FoxP3 PE-Cy7. Antibodies for the myeloid panel are as follows; CD45 APC-eFluor780, F4/80 eFluor450, CD11b APC, PD-L1 PE-Cy7, iNOS FITC, CD11c PE-Cy5.5, MHCII PE, Ly6G (Gr1) AF700. All antibodies were purchased from Ebiosciences except iNOS which was purchased from BD Bioscience. Both panels optimized a viability stain, FVS510 (BD Biosciences). For staining, cells were first stained with viability stain, rinsed, and blocked in Fc Block (BD Bioscience) per manufacturer's instructions. Extracellular staining was performed in 100 μL total staining volume protected from light for 30 mins at 4° C. Cells were then rinsed and fixed/permeabilized (Fix/Perm buffer eBioscience) at 4° C. overnight. The following day cells were rinsed and resuspended in Perm buffer (eBioscience), stained for intracellular markers (30 mins at 4° C.), rinsed, and resuspended in FACs buffer (PBS+2% FBS) for flow cytometry analysis using an LSR II flow cytometer (BD Bioscience). Flow cytometry analysis was performed using FlowJo v10 (FlowJo, LLC; Ashland, Oreg.).

Serum Cytokine Analysis—

Serum cytokine analysis optimized a 25-plex mouse cytokine/chemokine magnetic bead panel (EMD Millipore). Cytokines/chemokines assayed include G-CSF, GM-CSF, IFNγ, IL-1α, IL-1β, IL-2, IL-4, IL-5, IL-6, IL-7, IL-9, IL-10, IL-12 (p40), IL-12 (p70), IL-13, IL-15, IL-17, IP-10, KC, MCP-1, MIP-1a, MIP-1β, MIP-2, RANTES, and TNF-α. Samples were prepared via manufacturer's instructions and analysis was performed using Luminex LX200 (Luminex Corp. Austin, Tex.).

Experimental Design and Data Analysis—

For all experiments GraphPad Prism version 7.00 for Windows (La Jolla, Calif.) was used for graph generation and statistical analysis. Bi-comparisons (i.e. RFT vs. control) were statistically compared using an unpaired student's t-test. Multi-comparisons (i.e. tumor growth curves, serum cytokine analysis) were performed using a 2-way ANOVA with Bonferroni-corrected multi-comparison. For all comparisons, a p-value less than 0.05 was considered statistically significant and the following p-value representations were used throughout; *p<0.05, **p<0.01, ***p<0.001.

Results and Discussion

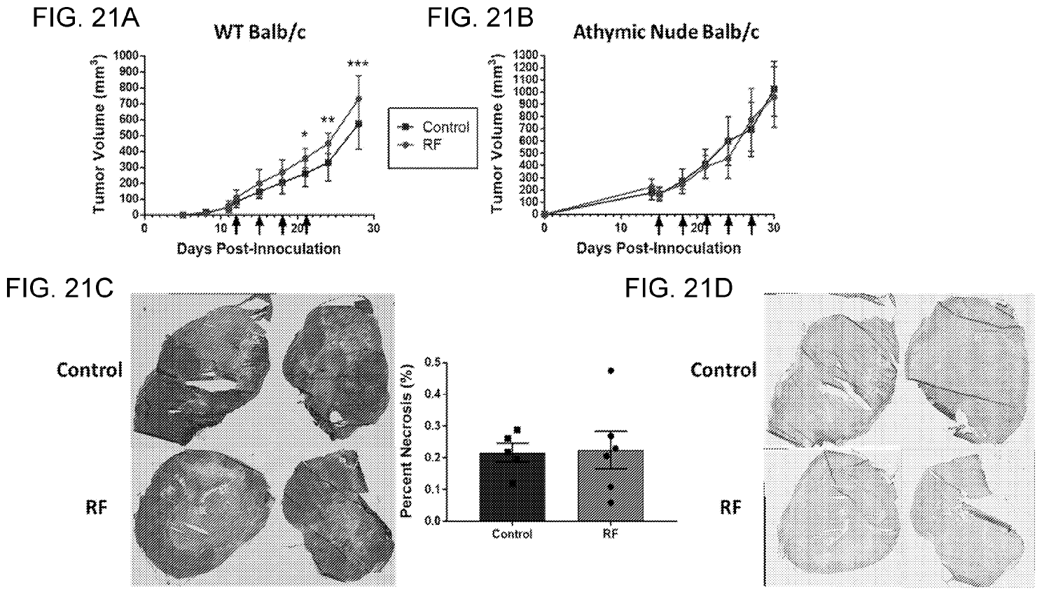

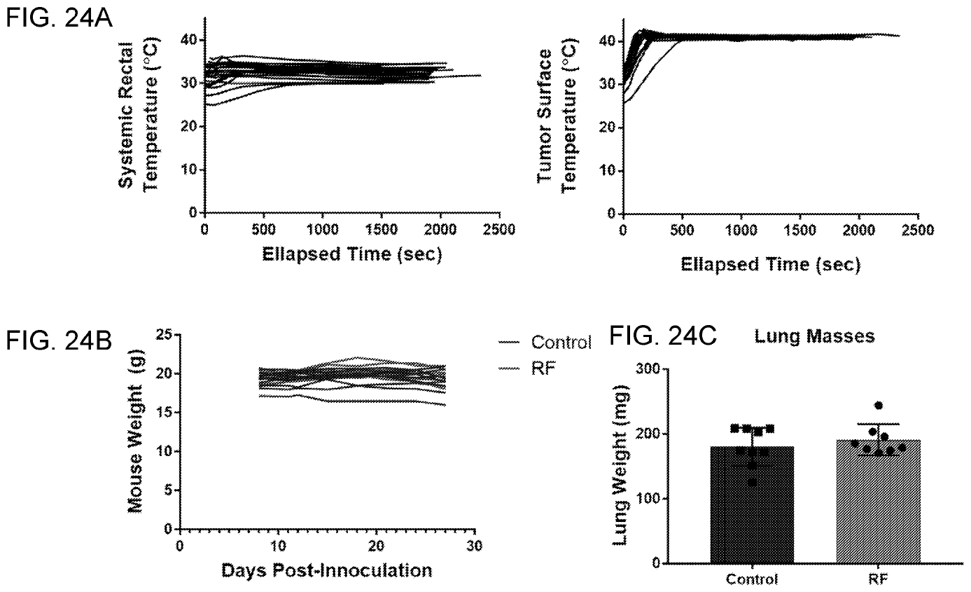

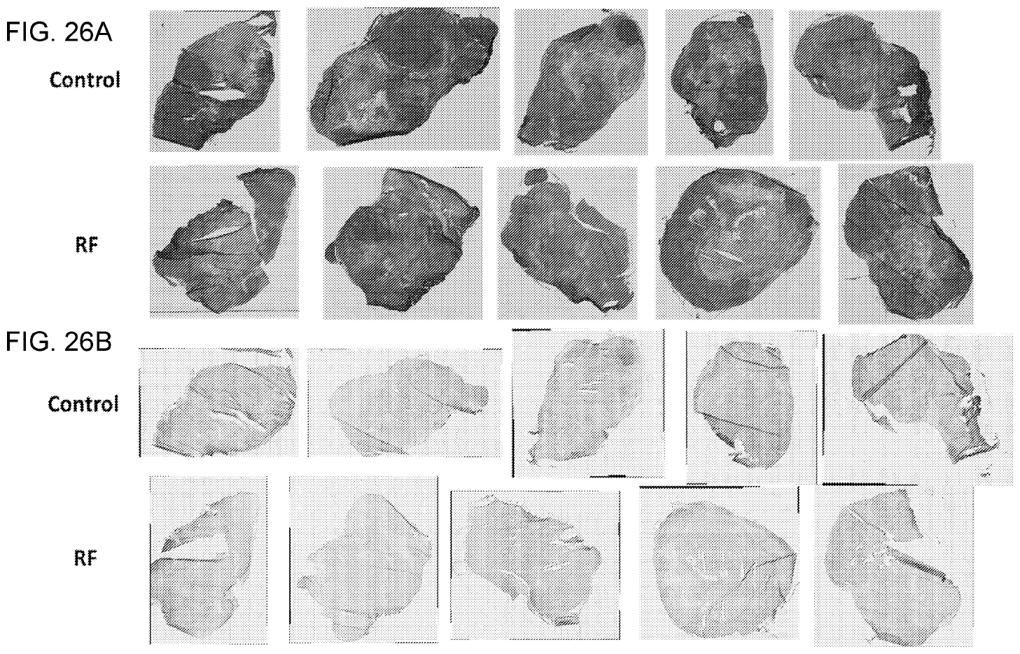

Using the optimized protocol developed previously (Ware, et al., 2017), mice bearing 4T1 tumors were treated with multiple doses of RFT. During treatment, tumor surface temperatures were increased to 41° C. and maintained there for 30 mins and systemic temperature of each mouse was measured using a rectally inserted fiber optic temperature probe. Using this method allowed for effective and consistent heating of tumor tissue and only minor systemic heating (FIGS. 20A-10C; FIG. 24A). Based on previous published work this would be consistent with a 40-41° C. internal tumor temperature, as RFT results in a less than 1° C. temperature differential between superficial and intra-tumoral temperature (Ware, et al., 2017). Control treated mice were placed on a heating pad under isoflurane to maintain similar systemic temperatures as the RFT mice, to better investigate the localized heating effects. RFT or control mice bearing established 4T1 tumors underwent numerous consecutive treatment every 3 days and tumor growth was measured throughout. Of interest, the RFT mice demonstrated a significant increase in tumor size compared to control mice after 4 total RF doses (FIG. 21A). This exaggeration in growth kinetics was not observed in immunodeficient athymic nude mice bearing similarly established 4T1 tumors following multiple RFT (FIG. 21B). Histologic analysis of these tumors revealed no major changes between the RF and control treated mice and quantification of tumor necrotic fraction revealed similar necrosis between both groups (FIG. 21C). In addition, Ki67 staining for tumoral proliferation showed similar levels between control and RF treated groups (FIG. 21D; see FIG. 26A-B for full set of images). Both subsets showed strong proliferation in extra-tumoral regions as expected per this aggressive 4T1 tumor model. Throughout treatment no observable toxicities were noted, and mouse weights further demonstrated the safety of this treatment regimen and modality (FIG. 24B). In addition, there were no signs of metastases at termination, and similar lung weights between RF and control mice further indicate no increase in metastatic potential following RFT (FIG. 24C). Collectively, these data indicate that RFT was inducing some form of inflammatory response within tumors that required functional T-cells and that this immunologic effect was primarily responsible for the change in tumor size after multiple doses of RFT.

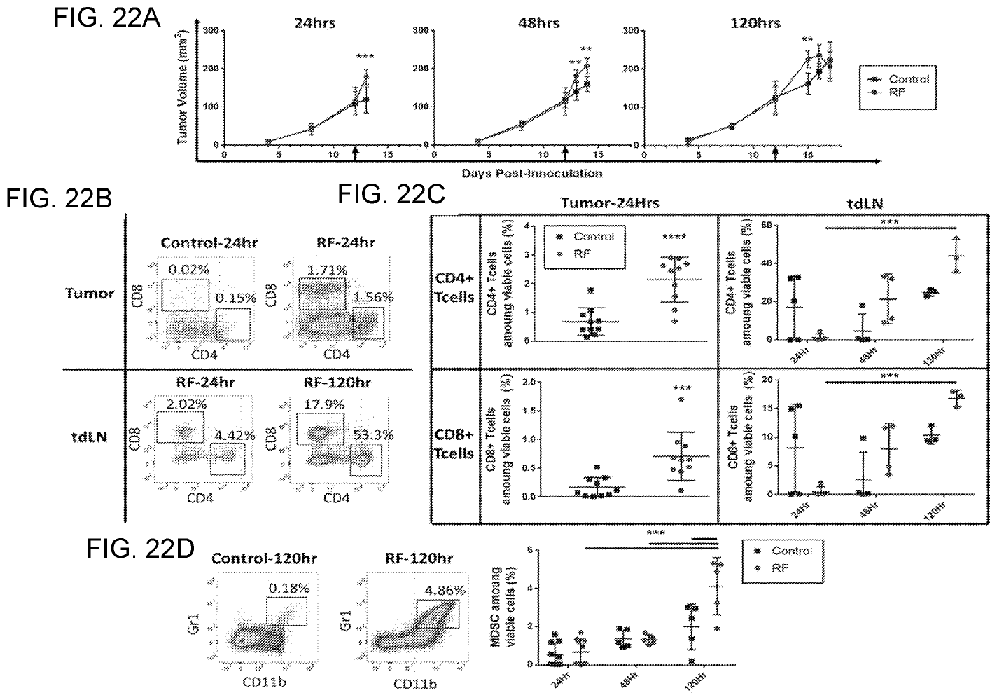

To better characterize the immunologic changes induced by RFT, an immune microenvironment analysis of tumor, spleen, blood, and tumor-draining inguinal lymph node was performed. For these studies immune competent Balb/c mice with established 4T1 tumors were treated with a single dose of RFT. Tissues were then processed and analyzed either 24, 48, or 120 hours after single-dose RFT. Similar to multi-RFT tumor growth characteristics, single-dose RFT tumors also displayed a transient growth response. RFT treated tumors showed a 50% increase in measured volume by 24 hours post-RFT which remained significantly larger than control tumors until 72 hours post-RFT, after which they returned to control tumor sizes (FIG. 22A). Immune microenvironment analysis further showed a transient tumoral influx of CD4+ and CD8+ T-cells which were increased by greater than 3- and 4-fold in RFT treated tumors 24 hours after treatment, respectively (FIGS. 22B, 3C). Increased T-cell levels returned to control levels by 48 hours after RFT. Analysis of tumor-draining inguinal lymph nodes showed an opposite effect as they appear entirely devoid of T-cells 24 hours post-RFT, but significantly increase more than 38- and 36-fold for CD4+ and CD8+ T-cells, respectively, by 120 hours post-RFT (FIGS. 22B, 22C). These data indicate that lymph-node dwelling T-cells are being drawn to the tumor rapidly after RFT is applied, rendering the lymph node devoid of lymphocytes. In addition, it would appear that the increases in T-cell trafficking following RFT are highly time-dependent, potentially peaking near 24 hours post-RFT. One potential factor that could be limiting further lymphocyte trafficking into tumors that received RFT is the 2-fold increase in MDSC at 120 hours post-RFT compared to control tumors (FIG. 22D). MDSC are well-known to employ a number of immunosuppressive mechanisms that can significantly inhibit the infiltration of lymphocytes into the tumor microenvironment (Junttila, et al., 2013). Thus, the increased MDSC may contribute to the build-up of T-cells within the lymph node as they inhibit further T-cell infiltration to the tumor microenvironment

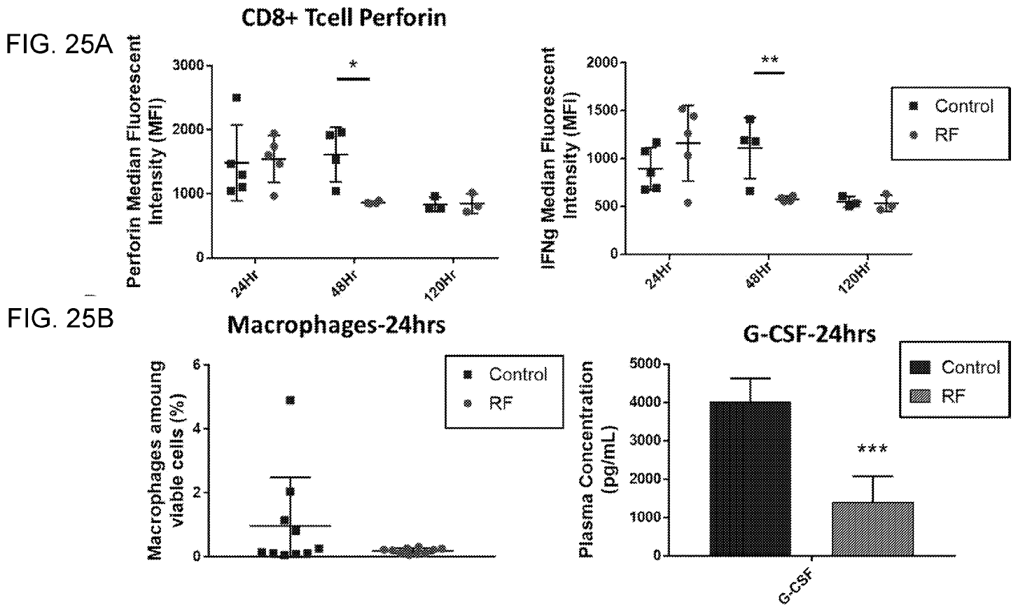

Splenic and hematologic changes appeared quite minor with only slight decreases in CD4+ T-cells, CD8+ T-cells, and macrophages in the blood at 24 hours post-RFT, which could be explained by the observed trafficking and other tumoral effects of RFT (Tables 1-4). In addition, the activation state of tumor dwelling CD8+ T-cells was also significantly reduced at 48 hours post-RFT, as they expressed lower levels of Perforin and IFNγ, two T-cell effector molecules (FIG. 25A). Nevertheless, the immunologic of RFT appear to be highly localized to the site of treatment, with only minor systemic alterations. Therapeutically this could provide an advantage to many other immunomodulatory treatments which have concerning systemic toxicities, usually involving systemic hyperactivation and concerns of cytokine storm. Overall, that data indicates that RFT can provide a highly localized and transient influx of lymphocytes which, if combined with other lymphocyte-focused immunotherapeutic strategies, will likely provide major combinatorial therapeutic benefit.

TABLE 1
Changes in primary immune cell subsets in the tumor, all as a
percent of total viable cells. Data presented as average (SEM).

Tumor-24 hrs

| | Control % | RF % |
|---|---|---|
| CD4+Tcell_{(CD45+/CD4+)} | 0.677 (0.152) | 2.141 (0.246) |
| CD8+Tcells_{(CD45+/CD8+)} | 0.169 (0.054) | 0.709 (0.134) |
| Treg_{(CD45+/CD4+/FoxP3+)} | 0.129 (0.040) | 0.230 (0.085) |
| NK Cell_{(CD45+/NK1.1+)} | 0.005 (0.001) | 0.005 (0.001) |
| Macrophage_{(CD45+, F4/80+, CD11b}^{Intermediate}₎ | 0.968 (0.482) | 0.389 (0.027) |
| DC_{(CD45+, F4/80-, CD11c+, MHCII}^{High}₎ | 1.874 (0.193) | 1.394 (0.235) |
| MDSC_{(CD45+, F4/80-, CD11c-, CD11b+, Gr1+)} | 0.512 (0.193) | 0.666 (0.215) |

Tumor-48 hrs

| | Control % | RF % |
|---|---|---|
| CD4+Tcell_{(CD45+/CD4+)} | 1.042 (0.586) | 0.420 (0.136) |
| CD8+Tcells_{(CD45+/CD8+)} | 0.248 (0.147) | 0.057 (0.033) |
| Treg_{(CD45+/CD4+/FoxP3+)} | 0.646 (0.471) | 0.215 (0.085) |
| NK Cell_{(CD45+/NK1.1+)} | 0.004 (0.002) | 0.002 (0.001) |
| Macrophage_{(CD45+, F4/80+, CD11b}^{Intermediate}₎ | 3.032 (1.356) | 2.206 (1.022) |
| DC_{(CD45+, F4/80-, CD11c+, MHCII}^{High}₎ | 1.892 (0.127) | 1.394 (0.198) |
| MDSC_{(CD45+, F4/80-, CD11c-, CD11b+, Gr1+)} | 1.366 (0.209) | 1.312 (0.110) |

| | Tumor-120 hrs | |
|---|---|---|
| | Control % | RF % |
| CD4+Tcell_{(CD45+/CD4+)} | 0.954 (0.339) | 0.435 (0.222) |
| CD8+Tcells_{(CD45+/CD8+)} | 0.204 (0.107 | 0.098 (0.052) |
| Treg_{(CD45+/CD4+/FoxP3+)} | 0.5782 (0.220) | 0.2742 (0.163) |
| NK Cell_{(CD45+/NK1.1+)} | 0.002 (0.001) | 0.001 (0.001) |
| Macrophage_{(CD45+, F4/80+, CD11b}^{Intermediate}₎ | 4.384 (0.449) | 4.742 (0.555) |
| DC_{(CD45+, F4/80-, CD11c+, MHCII}^{High}₎ | 1.936 (0.510) | 2.652 (0.423) |
| MDSC_{(CD45+, F4/80-, CD11c-, CD11b+, Gr1+)} | 0.996 (0.225) | 1.288 (0.117) |

TABLE 2
Changes in primary immune cell subsets in the spleen, all as a
percent of total viable cells. Data presented as average (SEM).

| | Spleen 24-hrs | |
|---|---|---|
| | Control % | RF % |
| CD4+Tcell_{(CD45+/CD4+)} | 4.620 (0.907) | 5.703 (1.583) |
| CD8+Tcells_{(CD45+/CD8+)} | 1.869 (0.500) | 2.107 (0.683) |
| Treg_{(CD45+/CD4+/FoxP3+)} | 0.148 (0.051) | 0.220 (0.080) |
| NK Cell_{(CD45+/NK1.1+)} | 0.280 (0.091) | 0.251 (0.084) |
| Macrophage_{(CD45+, F4/80+, CD11b}^{Intermediate}₎ | 2.536 (0.698) | 2.838 (0.691) |
| DC_{(CD45+, F4/80-, CD11c+, MHCII}^{High}₎ | 1.304 (0.106) | 1.312 (0.068) |
| MDSC_{(CD45+, F4/80-, CD11c-, CD11b+, Gr1+)} | 20.014 (6.775) | 16.584 (6.030) |

Spleen 48-hrs

| | Control % | RF % |
|---|---|---|
| CD4+Tcell_{(CD45+/CD4+)} | 4.238 (1.406) | 4.344 (0.289) |
| CD8+Tcells_{(CD45+/CD8+)} | 1.840 (0.596) | 1.448 (0.145) |
| Treg_{(CD45+/CD4+/FoxP3+)} | 0.095 (0.037) | 0.210 (0.022) |
| NK Cell_{(CD45+/NK1.1+)} | 0.700 (0.128) | 0.856 (0.079) |
| Macrophage_{(CD45+, F4/80+, CD11b}^{Intermediate}₎ | 3.898 (0.495) | 4.422 (0.439) |
| DC_{(CD45+, F4/80-, CD11c+, MHCII}^{High}₎ | 1.260 (0.200) | 1.44 (0.079) |
| MDSC_{(CD45+, F4/80-, CD11c-, CD11b+, Gr1+)} | 46.54 (4.766) | 43.12 (2.541) |

| | Spleen 120-hrs | |
|---|---|---|
| | Control % | RF % |
| CD4+Tcell_{(CD45+/CD4+)} | 4.380 (0.780) | 5.158 (0.766) |
| CD8+Tcells_{(CD45+/CD8+)} | 2.206 (0.410) | 2.416 (0.377) |
| Treg_{(CD45+/CD4+/FoxP3+)} | 0.159 (0.036) | 0.244 (0.071) |
| NK Cell_{(CD45+/NK1.1+)} | 0.438 (0.062) | 0.538 (0.082) |
| Macrophage_{(CD45+, F4/80+, CD11b}^{Intermediate}₎ | 2.996 (0.418) | 2.896 (0.276) |
| DC_{(CD45+, F4/80-, CD11c+, MHCII}^{High}₎ | 0.860 (0.241) | 1.050 (0.094) |
| MDSC_{(CD45+, F4/80-, CD11c-, CD11b+, Gr1+)} | 35.980 (9.932) | 43.200 (3.455) |

TABLE 3
Changes in primary immune cell subsets in the tumor draining inguinal lymph node,
all as a percent of total viable cells. Data presented as average (SEM).

tdLN 24-hrs

| | Control % | RF % |
|---|---|---|
| CD4+Tcell_{(CD45+/CD4+)} | 17.067 (7.287) | 1.149 (0.824) |
| CD8+Tcells_{(CD45+/CD8+)} | 8.115 (3.428) | 0.462 (0.390) |
| Treg_{(CD45+/CD4+/FoxP3+)} | 2.707 (1.510) | 0.123 (0.085) |
| NK Cell_{(CD45+/NK1.1+)} | 0.085 (0.021) | 0.299 (0.127) |
| Macrophage_{(CD45+, F4/80+, CD11b}^{Intermediate}₎ | 0.080 (0.034) | 0.588 (0.321) |
| DC_{(CD45+, F4/80-, CD11c+, MHCII}^{High}₎ | 0.601 (0.264) | 0.394 (0.197) |
| MDSC_{(CD45+, F4/80-, CD11c-, CD11b+, Gr1+)} | 0.126 (0.027) | 0.492 (0.154) |

tdLN 48-hrs

| | Control % | RF % |
|---|---|---|
| CD4+Tcell_{(CD45+/CD4+)} | 4.68 (4.374) | 21.398 (6.516) |
| CD8+Tcells_{(CD45+/CD8+)} | 2.545 (2.415) | 7.937 (2.223) |
| Treg_{(CD45+/CD4+/FoxP3+)} | 0.638 (0.584) | 1.220 (0.380) |
| NK Cell_{(CD45+/NK1.1+)} | 0.258 (0.084) | 0.058 (0.013) |
| Macrophage_{(CD45+, F4/80+, CD11b}^{Intermediate}₎ | 0.059 (0.025) | 0.043 (0.008) |
| DC_{(CD45+, F4/80-, CD11c+, MHCII}^{High}₎ | 0.191 (0.140) | 0.288 (0.046) |
| MDSC_{(CD45+, F4/80-, CD11c-, CD11b+, Gr1+)} | 0.578 (0.207) | 0.064 (0.003) |

| | tdLN 120-hrs | |
|---|---|---|
| | Control % | RF % |
| CD4+Tcell_{(CD45+/CD4+)} | 24.767 (1.073) | 44.000 (5.059) |
| CD8+Tcells_{(CD45+/CD8+)} | 10.32 (0.841) | 16.767 (0.809) |
| Treg_{(CD45+/CD4+/FoxP3+)} | 3.033 (0.462) | 2.660 (0.535) |
| NK Cell_{(CD45+/NK1.1+)} | 0.068 (0.042) | 0.057 (0.021) |
| Macrophage_{(CD45+, F4/80+, CD11b}^{Intermediate}₎ | 0.033 (0.009) | 0.019 (0.006) |
| DC_{(CD45+, F4/80-, CD11c+, MHCII}^{High}₎ | 0.270 (0.099) | 0.323 (0.131) |
| MDSC_{(CD45+, F4/80-, CD11c-, CD11b+, Gr1+)} | 0.189 (0.076) | 0.160 (0.036) |

TABLE 4
Changes in primary immune cell subsets in the blood, all as a
percent of total viable cells. Data presented as average (SEM).

Blood 24-hrs

| | Control % | RF % |
|---|---|---|
| CD4+Tcell_{(CD45+/CD4+)} | 1.454 (0.498) | 0.752 (0.139) |
| CD8+Tcells_{(CD45+/CD8+)} | 0.502 (0.175) | 0.250 (0.050) |
| Treg_{(CD45+/CD4+/FoxP3+)} | 0.304 (0.079) | 0.286 (0.199) |
| NK Cell_{(CD45+/NK1.1+)} | 0.026 (0.017) | 0.007 (0.002) |
| Macrophage_{(CD45+, F4/80+, CD11b}^{Intermediate}₎ | 0.538 (0.216) | 0.193 (0.062) |
| DC_{(CD45+, F4/80-, CD11c+, MHCII}^{High}₎ | 0.017 (0.005) | 0.009 (0.001) |
| MDSC_{(CD45+, F4/80-, CD11c-, CD11b+, Gr1+)} | 15.26 (6.839) | 6.55 (0.870) |

Blood 48-hrs

| | Control % | RF % |
|---|---|---|
| CD4+Tcell_{(CD45+/CD4+)} | 0.682 (0.159) | 0.988 (0.106) |
| CD8+Tcells_{(CD45+/CD8+)} | 0.206 (0.048) | 0.300 (0.029) |
| Treg_{(CD45+/CD4+/FoxP3+)} | 0.290 (0.164) | 0.180 (0.097) |
| NK Cell_{(CD45+/NK1.1+)} | 0.005 (0.002) | 0.006 (0.001) |
| Macrophage_{(CD45+, F4/80+, CD11b}^{Intermediate}₎ | 0.242 (0.056) | 0.226 (0.028) |
| DC_{(CD45+, F4/80-, CD11c+, MHCII}^{High}₎ | 0.015 (0.003) | 0.014 (0.002) |
| MDSC_{(CD45+, F4/80-, CD11c-, CD11b+, Gr1+)} | 11.230 (1.828) | 7.808 (1.089) |

Blood 120-hrs

| | Control % | RF % |
|---|---|---|
| CD4+Tcell_{(CD45+/CD4+)} | 1.168 (0.186) | 1.012 (0.115) |
| CD8+Tcells_{(CD45+/CD8+)} | 0.416 (0.061) | 0.376 (0.039) |
| Treg_{(CD45+/CD4+/FoxP3+)} | 0.854 (0.190) | 0.560 (0.159) |
| NK Cell_{(CD45+/NK1.1+)} | 0.023 (0.007) | 0.075 (0.033) |
| Macrophage_{(CD45+, F4/80+, CD11b}^{Intermediate}₎ | 1.900 (1.413) | 0.632 (0.340) |
| DC_{(CD45+, F4/80-, CD11c+, MHCII}^{High}₎ | 0.012 (0.004) | 0.016 (0.003) |
| MDSC_{(CD45+, F4/80-, CD11c-, CD11b+, Gr1+)} | 10.580 (3.256) | 7.216 (1.962) |

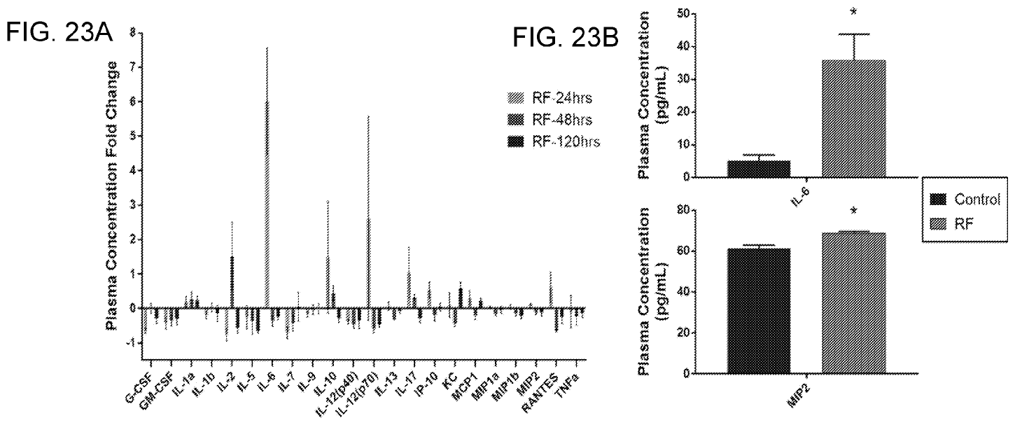

Because of the vast amount of previous evidence suggesting that key pro-inilammatory cytokine and chemokine expression following febrile hyperthermia promote T-cell infiltration (Mikucki, et al., 2013; Evans, et al., 2001), we investigated whether RFT treatment induced any significant changes in serum cytokine levels. Blood collected at 24, 48, and 120 hours after a single RFT were compared across a pro-inflammatory panel of 25 cytokines and chemokines. Similar to previous results, there was a highly transient cytokine response, with noted increases 24 hours after RFT′ in a number of proinflammatory cytokines including IL-6, IL-17, IL-12(p70), MIP2, and RANTES (FIG. 23A). appeared the most drastically, elevated, with a 6-fold increase induced 24-hours after a single dose of RFT (FIG. 23B). Despite the negative effects that IL-6 is known to induce in tumors, previous evidence would suggest that IL-6 alters its function under period of hyperthermic stress and strongly promotes T-cell activation and enhanced infiltration (Evans, et al., 2001). A more modest, though significant, increase was also noted in MIP2 (i.e. CXCL2), a potent chemotactic for leukocytes (FIG. 23B). This along with the other modest increases in chemokines such as RANTES (i.e. CXCL5) and IP-10 (i.e. CXCL10) could collectively contribute to the enhanced infiltration of T-cells by 24-hours post-RFT. On the other hand, G-CSF, a cytokine commonly released by endothelium and macrophages, underwent a 2.5-fold reduction 24-hours after RFT. This observation was further supported by the microenvironment analysis which showed that intra-tumoral macrophage were almost entirely depleted 24 hours post-RFT (FIG. 25B). Since this depletion was not associated with increased trafficking to the draining lymph node, this would suggest that tumor dwelling macrophages may have a lower tolerance for hyperthermia conditions resulting in their depletion. Interestingly, all elevated cytokines and chemokines appear to return to baseline levels by 48 hours post-RFT, with the exception of IL-2 that modestly increases at 48 hours post-RFT. The transient nature of the cytokine and chemokines directly corroborates our microenvironment analysis which showed the most potent intratumoral infiltration effects at 24 hours post-RFT. Overall, these data further suggest a transient immunologic effect induced by RFT, and suggest that the enhanced T-cell tumoral infiltration could be due at least in part by increased expression of a number of proinflammatory cytokines and leukocyte attractive chemokines.

### Significance of Certain Embodiments

With the emergence of immunotherapy as a promising cancer treatment modality, there remains an evident need for immunomodulatory strategies capable of promoting effector immune cell trafficking and infiltration in solid tumors. The use of non-invasive RFT, despite previously having been shown to promote effective and safe intratumoral hyperthermia, has never been fully investigated for its immunomodulatory potential. Herein is described the immunomodulatory effects of RFT on a 4T1 murine breast tumor model. RFT-induced hyperthermia drives a highly localized and transient intra-tumoral inflammatory response. This effect promoted a 3- and 4-fold increase in the intra-tumoral infiltration of CD4+ and CD8+ T-cells, respectively, 24 hours after a single RFT was applied. This effect was further manifested as a transient tumoral growth effect, in which tumors appeared exaggerated in size up to 72 hours after a single RFT was applied. In specific embodiments, the increased lymphocytes trafficking is associated with inflammation and T-cell trafficking, as various cytokines associated with inflammation and T-cell trafficking were elevated in RFT mice, especially IL-6. Despite these promising effects however, treatment efficacy of RET alone was not observed. This limitation is likely contributed by numerous factors including: 1) the lack of tumor-specific and effector T-cell generation, 2) increased trafficking of immunosuppressive MDSC populations after RFT, and 3) T-cell exhaustion or lack of critical lymphocyte signaling. This highlights the need to further combine RFT with other immunotherapeutic strategies that promote lymphocyte activation, enhance T-cell tumor specificity, and prevent exhaustion; such as immune checkpoint inhibitors, chimeric antigen receptor (CAR) T-cells, and cancer vaccine strategies. Overall, these data demonstrate that non-invasive RFT may provide an effective immunomodulatory method in solid tumor cancers, and its combination with other immunotherapeutic strategies will provide significant combinatorial therapeutic benefit.

Moreover, the scope of the present application is not intended to be limited to the particular embodiments of the process, machine, manufacture, composition of matter, means, methods and steps described in the specification.

1. A method of treating an individual for cancer, comprising the step of delivering to the individual a therapeutically effective amount of radiofrequency therapy (RF) and immunotherapy.

2. The method of claim 1, wherein the immunotherapy comprises one or more immune checkpoint inhibitors, therapeutic vaccine targeting tumor antigens; innate immune-stimulating molecules or biologics; adaptive immune-stimulating molecules or biologics; monoclonal antibodies or other agents targeting positive immune costimulatory molecules; adoptive cellular therapy; other drugs that influence immune cell function to enhance anti-cancer activity; chimeric antigen receptor (CAR) T-cells; cancer vaccine; or a combination thereof.

3. The method of claim 2, wherein the immune checkpoint inhibitor targets CTLA-4, PD1, PD-L1, TIM-3, B7-H3, IDO, or a combination thereof.

4. The method of claim 1, wherein the immunotherapy comprises an INOS inhibitor.

5. The method of claim 4, wherein the INOS inhibitor is LNIL, L-NMMA 1400 W dihydrochloride, AR-C 102222, AMT hydrochloride, S-Isopropylisothiourea hydrobromide, Aminoguanidine hydrochloride, BYK 191023 dihydrochloride, EIT hydrobromide, (S)-Methylisothiourea sulfate or a combination thereof.

6. The method of claim 1, wherein the immunotherapy comprises one or more immune-stimulating molecules.

7. The method of claim 6, wherein the immune-stimulating molecule is GITR, OX-40, IL-2, IL-12, IL-18, IFNα, IL-11, GM-CSF, G-CSF, or other positive costimulatory molecules that may be targeted by agonist monoclonal antibodies or other means.

8. The method of claim 1, wherein the RF and the immunotherapy are delivered to the individual at the same or different times.

9. The method of claim 8, wherein the RF is delivered to the individual before the immunotherapy.

10. The method of claim 8, wherein the RF is delivered to the individual after the immunotherapy.

11. The method of claim 1, wherein the RF is delivered to the individual multiple times.

12. The method of claim 1, wherein the immunotherapy is delivered to the individual multiple times.

13. The method of claim 1, wherein the individual is given a therapy other than the RF and the immunotherapy for the cancer.

14. The method of claim 13, wherein the other therapy is another immunotherapy, surgery, chemotherapy, radiation, hormone therapy, or a combination thereof.

15. The method of claim 13, wherein the individual is given surgery prior to or after the delivery of the RF and immunotherapy.

16. The method of claim 1, wherein the RF is given to the individual for at least 5, 10, 15, 20, 30, 35, 40, 45, 50, 55, or 60 minutes in duration.

17. The method of claim 1, wherein the temperature that is generated at a desired location to which the radiofrequency is directed is between 37° C. and 45° C.

18. A method of inducing an intra-tumoral inflammatory response at a tumor site in an individual, comprising the step of providing to the individual a therapeutically effective amount of radiofrequency therapy (RF) and immunotherapy such that the immunotherapy in combination with the RF therapy will exhibit an additive or synergistic effect at the tumor site of the individual.
